(19) United States
(12) Patent Application Publication
(10) Pub. No.: US 2013/0266664 A1
(43) Pub. Date: Oct. 10, 2013

(54) FACILE METHOD FOR CROSSLINKING AND INCORPORATING BIOACTIVE MOLECULES INTO ELECTROSPUN FIBER SCAFFOLDS

(71) Applicants: Hu Yang, Richmond, VA (US); Gary L. Bowlin, Richmond, VA (US); Alpana Dongargaonkar, Richmond, VA (US)

(72) Inventors: Hu Yang, Richmond, VA (US); Gary L. Bowlin, Richmond, VA (US); Alpana Dongargaonkar, Richmond, VA (US)

(73) Assignee: VIRGINIA COMMONWEALTH UNIVERSITY, Richmond, VA (US)

(21) Appl. No.: 13/996,161

(22) PCT Filed: Dec. 20, 2011
(86) PCT No.: PCT/US11/66071

§ 371 Date: Jun. 20, 2013

Related U.S. Application Data
(60) Provisional application No. 61/424,835, filed on Dec. 20, 2010.

(51) Int. Cl. A61K 47/42

(52) U.S. Cl. CPC A61K 47/42
USPC 424/618; 530/354; 514/774; 427/508; 427/2.31

(57) ABSTRACT

Electrospun scaffolds crosslinked with acrylates and methods of making the same are provided. Because the cross-linking linking is carried out under mild conditions, biologically active agents are incorporated into the scaffolds in a facile manner.

### BACKGROUND OF THE INVENTION

1. Field of the Invention

The invention generally relates to crosslinked electrospun scaffolds and methods of making the same. In particular, the invention provides electrospun scaffolds that are cross-linked using acrylates and which incorporate biologically active agents.

2. Background of the Invention

Electrospinning has been widely used to create fiber scaffolds for tissue engineering and other applications, with both synthetic and natural polymers being used to produce the scaffolds. Unfortunately, however, electrospun scaffolds are not always sufficiently robust to be used for a desired purpose, and/or may not exhibit a desired rate of dissolution in applications which are transient in nature, e.g. where resorption of the scaffold is required or desirable. To modulate the mechanical properties and degradation rates of electrospun scaffolds, chemical crosslinking has been adopted. Unfortunately, many chemical crosslinking agents are highly toxic and unsuitable for use in a scaffold that is to be used in a biological system.

In addition, for some applications, it is advantageous to include biologically active therapeutic agents in electrospun scaffolds. For example, the addition of growth factors, anti-microbial agents, etc. to electrospun materials used for wound treatment can be highly beneficial. However, care must be taken to incorporate such active agents in a manner that allows them to retain their bioactivity, and which permits delivery of the agents from the electrospun material to a site of action in a safe and useful manner. For example, extremely rapid release of the agent maybe desired for some applications while slow release may be advantageous for others. Crosslinking of scaffolds that are designed to deliver biologically active agents is highly desirable as a way to modulate the scaffold's properties. Unfortunately, the conditions for crosslinking are typically harsh, and the activity of therapeutic agents can be compromised if they are exposed to such conditions while being incorporated into electrospun material, either during electrospinning or crosslinking.

There is an ongoing need in the art to address these and other issues in order to improve the properties of electrospun materials and the delivery of biologically active therapeutic agents using electrospun materials.

### SUMMARY OF THE INVENTION

The present invention provides electrospun scaffolds or matrices that are covalently crosslinked using photoreactive acrylates. While acrylates are widely used to cross-link hydrogel materials, their use to crosslink electrospun materials has not been previously described. The use of acrylates is advantageous compared to the use of previously employed crosslinking agents because they are non-toxic and thus safe to use in products designed for use in living systems. The use of acrylates advantageously permits the tailoring of multiple properties of electrospun materials (e.g. porosity, tensile strength, degradation rate, etc.) over a wide range of values, while maintaining and reinforcing the material's structure. In addition, since crosslinking is a separate procedure from electrospinning, this method allows encapsulation of bioactive molecules into electrospun fiber matrices in a noninvasive, nondestructive manner during the crosslinking process. Reaction conditions for photoactivation of acrylates are relatively mild, so biologically active agents can be incorporated (e.g. encapsulated, fixed or trapped) within electrospun materials during the crosslinking process without destroying or compromising their biological activity.

As described herein, photoactivatable acrylates are efficient agents for crosslinking fiber matrices and for securing biologically active agents within the matrices. This method of crosslinking permits the production of electrospun fiber materials which exhibit a wide spectrum of physical and biological properties, including porosity, permeability, mechanical modulus, strength, biodegradability, biocompatibility, etc. The crosslinked scaffolds are used in a variety of applications, including wound dressings, scaffolds for tissue growth and engineering, and implantable devices to provide support and/or to deliver biologically active agents to a site of interest, e.g. in a living organism.

It is an object of this invention to provide an electrospun scaffold that is crosslinked with an acrylate. The acrylate that is used may be, for example, polyethylene glycol (PEG) diacrylate. In one embodiment, the electrospun scaffold further comprises silver associated with the electrospun scaffold. In another embodiment, the electrospun scaffold comprises gelatin and/or dendrimers, and, optionally, at least one bioactive agent is associated with the electrospun scaffold.

The invention further comprises a material, comprising electrospun fibers selected from a plurality of natural and synthetic fibers or blends, said plurality of fibers configured as a mat, wherein individual fibers within said plurality of electrospun fibers are crosslinked by an acrylate. In one embodiment, one or more dendrimers bonded to one or more fibers of said plurality of fibers. In another embodiment, at least one bioactive agent is associated with the material.

The invention also provides a method of making a cross-linked fiber scaffold. The method comprises the steps of i) electrospinning a solution comprising at least one polymer to form a fiber scaffold; ii) associating a photoreactive acrylate with the fiber scaffold; and iii) activating said photoreactive acrylate by exposing said photoreactive acrylate to a source of radiation (e.g. ultraviolet light, etc.), wherein the step of activating causes chemical crosslinking of fibers in the fiber scaffold via activated photoreactive acrylate. In some embodiments, the method further comprises the step of associating at least one biologically active agent with the fiber scaffold prior to the step of activating. In another embodiment, the at least one polymer is selected from the group consisting of gelatin, at least one dendrimer, synthetic or natural polymers, and combinations thereof. In some embodiment, the at least one polymer includes gelatin and at least one dendrimer, for example, a polyamidoamine (PAMAM) dendrimer. In some embodiments, the photoreactive acrylate is polyethylene glycol (PEG) diacrylate. In some embodiments, the biologically active agent is selected from the group consisting of an antimicrobial agent, biologically active peptides and proteins, nucleic acids (e.g., DNA, siRNA, shRNA, etc), drugs, cells, cytokines, lipids, antibodies, vectors, adhesives, permeation enhancers, metals, inorganic agents, imaging and contrast agents, or micro- or nano-particles carrying the agents mentioned herein, etc.

The invention further provides a method of incorporating at least one biologically active agent into an electrospun scaffold. The method comprises the steps of 1) associating the at least one biologically active agent with said electrospun scaffold; and 2) crosslinking the electrospun scaffold with acrylate.

The invention also provides a method of releasing at least one biologically active agent at a site in or on a subject in need thereof. The method comprises the steps of 1) associating the at least one biologically active agent with an electrospun scaffold; 2) crosslinking the electrospun scaffold with acrylate; and 3) contacting the site with the crosslinked electrospun scaffold in a manner that permits release of the at least one biologically active agent at the site. For example, the electrospun scaffold may be placed directly on the site (e.g. a wound) so that biological fluid from the site comes into contact with at least a portion of the active agent, and/or so that the electrospun fibers of the scaffold dissolve or disintegrate in the biological fluid, thereby releasing the active agent. Alternatively, the site may be moistened or wetted to permit, initiate or foster release of the active agent with or without breakdown of the scaffold.

### BRIEF DESCRIPTION OF THE DRAWINGS

FIG. 1. A, a schematic representation of a crosslinked electrospun scaffold; B, reaction mechanism-conjugation of dendrimer to gelatin.

FIG. 2. SEM images of the non-crosslinked scaffolds. (The white block arrows indicate the notation of the scale bar of 10 μm).

FIG. 3. SEM images of the scaffolds crosslinked by the solution method. (The white block arrows indicate the notation of the scale bar of 10 μm).

FIG. 4. Fiber diameter of scaffolds (non-crosslinked and crosslinked by solution method).

FIG. 5A-C. Graphical representation of stress, strain and modulus (A: Stress, B: Strain, C: Modulus).

FIG. 6. Porosity of the scaffolds crosslinked by the solution method.

FIG. 7. Permeability of the scaffolds crosslinked by the solution method.

FIG. 8. Pore size of the scaffolds crosslinked by the solution method.

FIG. 9. Swelling of the scaffolds crosslinked by the solution method (in SWF at room temperature).

FIGS. 10A and B. Degradation of the scaffolds crosslinked by the solution method. A, scaffold S1; B, scaffold S5.

FIG. 11. Silver release kinetics.

FIG. 12A-N. Uncrosslinked and crosslinked gelatin fiber scaffolds. A, uncrosslinked, 10 μm; B, uncrosslinked, 100 μm; C, uncrosslinked, 30 min incubation, 5% (v/v) PEG diacrylate, 0.2% (w/v) DMPA, 10 μM; D, uncrosslinked, 30 min incubation, 5% (v/v) PEG diacrylate, 0.2% (w/v) DMPA, 100 μm; E, crosslinked, 12 hr incubation; 5% (v/v) PEG diacrylate, 0.2% (w/v) DMPA, 10 μm; F, crosslinked, 12 hr incubation; 5% (v/v) PEG diacrylate, 0.2% (w/v) DMPA, 100 μm; G, crosslinked, 24 hr incubation; 5% (v/v) PEG Diacrylate, 0.2% (w/v) DMPA, 10 μm; H, crosslinked, 24 hr incubation; 5% (v/v) PEG diacrylate, 0.2% (w/v) DMPA, 100 μm; I, crosslinked, 10% (v/v) PEG diacrylate, 0.4% (w/v) DMPA, 10 μm; J, crosslinked, 10% (v/v) PEG Diacrylate, 0.4% (w/v) DMPA, 100 μm; K, crosslinked, 20% (v/v) PEG diacrylate, 0.8% (w/v) DMPA, 10 μm; L, crosslinked, 20% (v/v) PEG diacrylate, 0.8% (w/v) DMPA, 100 μm; M, crosslinked, 40% (v/v) PEG diacrylate, 1.6% (w/v) DMPA, 10 μm; N, crosslinked, 40% (v/v) PEG Diacrylate, 1.6% (w/v) DMPA, 100 μm.

FIG. 13. Fiber diameter as a function of crosslinker concentration.

FIG. 14. Fiber diameter as a function of incubation time.

FIGS. 15A and B. Stress and strain as a function of incubation time. A, peak stress; B, strain at break.

FIGS. 16A and B. Stress and strain as a function of crosslinker concentration. A, peak stress; B, strain at break.

FIG. 17. In vitro degradation in DMEM+10% FBS as a function of incubation time.

FIG. 18. In vitro degradation in simulated salivary fluid as a function of incubation time.

FIG. 19. In vitro degradation in DMEM control as a function of incubation time.

FIG. 20. In vitro degradation in DMEM+10% FBS as a function of crosslinker concentration.

FIG. 21. In vitro degradation in simulated salivary fluid as a function of crosslinker concentration.

FIG. 22. In vitro degradation in DMEM control as a function of crosslinker concentration.

FIG. 23. Porosity as a function of concentration.

FIG. 24. Porosity as a function of incubation time.

FIG. 25. Comparison of time dependent swelling kinetics.

### DETAILED DESCRIPTION

According to the invention, photoreactive acrylates are advantageously used to crosslink electrospun fiber matrices in order to provide additional tensile strength and structural stability to the material. Crosslinking serves to “lock” the fibers of an electrospun matrix/scaffold into place, adding increased support and rigidity to the structure. This can improve the ease of handling and manipulating the scaffold, and increase its ability to maintain its shape and integrity during use, e.g. in a biological system. At the same time, varying degrees of porosity may be introduced by varying the amount, degree or type (e.g. the chemical nature) of the crosslinking, thereby modulating the ingress and egress of materials into and out of the scaffold (e.g. therapeutic agents, cells, etc), as described in detail herein. Further, by varying the degree or type of crosslinking, the biodegradability of the scaffold also can be varied.

In an exemplary embodiment, this method has been demonstrated by making crosslinked fiber scaffolds from gelatin and/or dendrimer-gelatin hybrids, or from alginate, chitosan, chitin, collagen, fibrinogen or from blends of these materials in unmodified form and/or coupled to dendrimer. Dendrimer-gelatin hybrid fiber constructs integrate advantages of both dendrimers and fibers for wound healing and drug delivery. The use of gelatin is desirable, for example, because of its usefulness for denial wound healing. Dendrimer-gelatin hybrid fiber constructs can be fabricated to contain various loading forms of dendrimers with a wide range of structural characteristics. The resultant fiber scaffolds are then crosslinked with a photoreactive acrylate species such as photoreactive PEG diacrylate to achieve stable constructs. Further, during crosslinking, bioactive molecules and/or therapeutics can be introduced into the matrix in a mild, non-destructive manner. The resulting crosslinked electrospun scaffolds have a variety of applications, both in vitro and in vivo, as described herein.

The invention provides electrospun materials, which may be referred to herein as scaffolds, matrices, supports, mats, etc., that are crosslinked using photoreactive (photoactivatable) acrylates. The electrospun materials typically comprise fibers with dimensions in about the nanometer to micrometer range, e.g. dimensions may be measured in millimeters, micrometers, nanometers, or Angstroms. Those of skill in the art are familiar with electrospinning and various techniques of electrospinning, such as those described, for example, in the following issued US patents, the contents of each of which is hereby incorporated by reference in entirety: U.S. Pat. Nos. 8,052,407; 7,134,857; 7,592,277; 7,981,353; 7,858,837; 7,828,539; 7,737,131; 7,569,359; 7,390,760; 7,244,272; 6,592,623; 7,759,082; 7,374,774; and 7,615,373. Briefly, a polymer solution for electrospinning is loaded into a syringe (or other suitable delivery reservoir), and a positively charged electrode is attached to the needle of the syringe. The application of voltage results in production of an electric field which causes a drop of polymer solution at the tip of the needle to form a conical shape known as the “Taylor cone”. As the strength of the electric field increases, the liquid cone forms into an elongated jet which, as a result of solvent evaporation, forms long, thin fibers which are collected on a grounded collector or mandrel. The mandrel typically undergoes translation and/or rotation to foster deposition of the fibers so as to produce a matrix or scaffold of a desired size and shape. The resulting electrospun matrix can be further modified as desired, e.g. by cutting, coating, and/or by other manipulations, either before or after subsequent crosslinking with acrylates.

Many types of polymers may be used to form electrospun fibers in this manner, including but not limited to: polyurethane, polyester, polyolefin, polymethylmethacrylate, polyvinyl aromatic, polyvinyl ester, polyamide, polyimide, polyether, polycarbonate, polyacrilonitrile, polyvinyl pyrrolidone, polyethylene oxide, poly (L-lactic acid), poly (lactide-co-glycoside), polycaprolactone (PCL), polyphosphate ester, poly (glycolic acid), poly (DL-lactic acid), and some copolymers (e.g. PLA co-polymers of PGA and PLA (PEG-PLA), and dendritic PEG-PLA), polyesters, and native proteins such as collagen, gelatin, fibronectin, fibrinogen, recombinant proteins and other natural and synthetic proteins and peptide sequences); biomolecules such as DNA, silk (e.g. formed from a solution of silk fiber and hexafluoroisopropanol), chitosan and cellulose (e.g. in a mix with synthetic polymers); various polymer nanoclay nanocomposites; halogenated polymer solution containing a metal compounds (e.g. graphite); PEGylated synthetic polymers and natural polymers (e.g., collagen, alginate); memory polymers including block copolymers of poly(L-lactide) and polycaprolactone and polyurethanes, and/or other biostable polyurethane copolymers, and polyurethane ureas; linear poly(ethylenimine), grafted cellulose, poly(ethyleneoxide), and poly vinylpyrrolidone; solutions of polystyrene (μS) in a mixture of N,N-dimethyl formamide (DMF) and tetrahydrofuran (THF) poly(vinyl pyrrolidone) (PVP) composites; poly(L-lactide), poly(D,L-lactide), polyglycolide, polycaprolactone, polydioxanone, poly(trimethylene carbonate), poly(4-hydroxybutyrate), poly(ester amides) (PEA), polyurethanes, and copolymers thereof; various polyesters and acrylics; various colloidal dispersions; solutions with dispersed hydroxyapatite (HA) particles; polysulfone and a vinyl lactam polymers; dextrans; various charged nylons (e.g. nylon 66 for protein adhesion and other variants designed to adhere to RNA and DNA); nitrocellouse; dendritic poly(ethylene glycol-lactide); etc. These materials and electrospinning techniques and variants thereof (e.g. various applications of electrospun materials, various coatings, etc.) are described, for example, in issued U.S. Pat. Nos. 6,110,590; 7,887,772; 7,824,601; 7,794,219; 7,759,082; 7,615,373; 7,575,707; 7,374,774; 7,083,854; 6,787,357; 6,753,4541; and 6,592,623; and published US patent applications 20110150973; 20110148004; 20110143429; 20110140295; 20110135901; 20110130063; 20110123592; 20110092937; 20110091972; 20110079275; 20110072965; 20110064949; 20110052467; 20100310658; 20100291058; 20080159985; 20080038352; 20050192622; 20040116032; 20040009600; and 20030207638; the complete contents of each of which are hereby incorporated by reference, as are the references cited therein.

In some embodiments, the solution that is used comprises gelatin. In other embodiments, the solution that is used comprises dendrimers. In yet other embodiments, the solution comprises a mixture of gelatin and dendrimers.

As used herein “gelatin” refers to the glutinous material obtained from animal tissues by using various methods such as boiling. Gelatin is a mixture of peptides and proteins produced by partial hydrolysis of collagen extracted from the boiled crushed bones, skin, connective tissues, etc. of animals such as domesticated cattle, chicken, and pigs. In gelatin, the natural molecular bonds between individual collagen strands are broken down into a form that rearranges more easily.

As used herein “dendrimers” (from the Greek word “dendron” for tree), refers to a synthetic, three-dimensional molecule with repetitive branching parts and with nanometer-scale dimensions. Dendrimers typically comprise three components: a central core (e.g. a central chain of carbon atoms), an interior regular dendritic structure (the “branches”), and, optionally, an exterior surface with functional (reactive) surface groups. Dendrimers are typically highly symmetric around the single core, the regular branching structure often adopting an overall spherical three-dimensional morphology. Dendrimers are formed using a nano-scale, multistep fabrication process in which the single core is repeatedly capped with successive layers of branches, each step resulting in a new “generation” that has twice the complexity of the previous generation. Synonymous terms for dendrimer include arborols and cascade molecules. There are many properties of dendrimers that make them attractive for biomedical applications: (i) they are monodisperse macromolecules (consistent size and form); (ii) they have low polydispersity index; (iii) they are highly soluble and miscible due to their branched structure; (iv) drug molecules can be encapsulated in their central core or covalently attached to their surface groups; and (v) their structurally stable architecture permits controlled drug release Various types of dendrimers are known in the art and can be used in the practice of the invention, including but not limited to: polyamidoamine (PAMAM) dendrimers e.g. as described in US patent application 20110189291; poly(propyleneimine) (PPI) dendrimers, polylysine dendrimers, or any highly branched nanostructures with reactive functional surface groups e.g. amine, carboxylate, hydroxyl, etc.

According to the invention, a fiber matrix or scaffold is foamed and is subsequently crosslinked using photoreactive acrylates. As used herein, the term “acrylate” refers to the salts and esters of acrylic acid. They are also known as propenoates (since acrylic acid is also known as 2-propenoic acid). Acrylates contain vinyl groups, that is, two carbon atoms double bonded to each other, directly attached to a carbonyl carbon. A photoreactive or photoactivatable acrylate is an acrylate that, upon exposure to a suitable wavelength of light (usually ultraviolet light), forms a highly reactive species such as a free radical, which then reacts indiscriminately with atoms or groups of atoms in its surroundings, forming covalent chemical bonds and linking surrounding elements together in a mesh-like structure.

Exemplary acrylates that may be used in the practice of the invention include the following: trimethylolpropane triacrylate (TMPTA), trimethylolpropane polyoxyethylene triacrylates, urethane acrylates; alkoxy-PEG acrylate and methacrylate; acrylates; allyl acrylate, pentaerythritol triacrylate, ethylene glycol dimethacrylate (EDMA), trimethylolpropane triacrylate; asymmetric (meth)acrylates; hydroxyalkyl methacrylates; various diacrylates, triacrylates, and tetraacrylates; various bicyclic cyclopropaneacrylates and polyfunctional (meth)acrylates; polyethylene glycol diacrylate, polyethylene glycol dimethacrylate, propylene glycol diacrylate, butanediol diacrylate, pentanediol diacrylate, 1,6-hexanediol diacrylate, 1,6-hexanediol dimethacrylate, tetraethylene glycol dimethacrylate, glycerol diacrylate, tripropylene glycol diacrylate and polyester diacrylate; trifunctional monomers such as trimethylol propane triacrylate, pentaerythritol triacrylate, trimethylol propane trimethacrylate, tris(acryloxyethyl)isocyanurate, ethoxylated trimethylol propane triacrylate and propoxylated glycerol triacrylate; polyfuctional monomers such as ditrimethyolpropane tetraacrylate pentaerythritol tetraacrylate and dipentaerythritol hydroxy pentaacrylate; acrylates having two or more acrylate moieties such as highly alkoxylated (meth)acrylates, e.g. ethoxylated bisphenol-A-diacrylate, ethoxylated trimethylol propane triacrylate, tripropylene glycol diacrylate, alkoxylated acrylates, tetraethylene glycol diacrylate, neopentyl glycol propoxylate diacrylate and polyethylene glycol di(meth)acrylate; dendrimer coupled with PEG acrylate (e.g., PAMAM dendrimer conjugated with various amounts of PEG acrylate of various lengths; various photoreactive acrylates, etc. These and other suitable acrylate species are listed or described in the following issued US patents or published US patent applications, the complete contents of each of which are hereby incorporated by reference in entirety: U.S. Pat. Nos. 4,408,016; 4,900,126; 5,011,762; 6,083,660; 6,660,799; 6,881,858; 7,387,642; 8,034,254; and 20100311863. In some embodiments, the acrylate species is a polyethylene glycol (PEG) acrylate such as PEG diacrylate. The photoreacive acrylates may be used alone or in combination with other photoreactive acrylates or photoreactive species.

In some embodiments of the invention, one or more (i.e. at least one) biologically active and/or therapeutic agent is incorporated into the fiber scaffolds described herein, usually before or during crosslinking of the scaffolding fibers is carried out. The crosslinking thus serves to trap or lock the agents within the scaffold. The agents are typically held within the scaffold by non-covalent bonds, and may be simply sterically blocked from leaving the scaffold as a result of the crosslinking. Alternatively, the agents may be chemically bonded (either non-covalently, or covalently as a result of crosslinking) to the scaffolding. The active agents may be introduced into the scaffolding (prior to photoactivation) in any of many suitable ways that will occur to those of skill in the art. For example, the scaffolding may be soaked in a solution of such agents, or the agents may be sprayed or injected onto or into the scaffold, or the agents may be added to a crosslinking solution prior to photoactivation, etc.

Those of skill in the art will recognize that a wide variety of biologically active beneficial, therapeutic or prophylactic active agents can be incorporated into the crosslinked scaffolds of the invention. Any agent that can be sequestered within the scaffolding matrix, whether transiently or permanently, may be incorporated, so long as a sufficient (clinically useful) quantity of the agent is trapped within the matrix after crosslinking, and so long as the crosslinking process does not destroy the biological activity of interest of the agent, and so long as the agent is accessible during use in the application for which is it intended so that its biological effect can be exerted. In some embodiments, the agent, after administration to a subject and upon contact with a biological fluid of the subject, leaches or diffuses from the matrix into the fluid, where the agent is then available to act, e.g. by circulating throughout a biological system of the subject (e.g. the circulatory system), and then contacting and in some cases entering cells and/or tissues where its effect is exerted. In other embodiments, the active agent is remains largely associated with the scaffold after administration (e.g. is retained within or on the scaffolding) and is released chiefly as a result of degradation (breakdown, resorption, etc.) of the scaffolding. In other embodiments, the agent is largely retained within and/or on the scaffold and acts on cells which infiltrate or otherwise contact the scaffolding. Those of skill in the art will recognize that these mechanisms of action are not necessarily mutually exclusive, and that combinations of these may occur in order to allow or permit the agent to contact a targeted biological environment (molecule, cells, tissue, etc.) and exert its effect thereon.

Exemplary active agents include those described, for example in U.S. Pat. Nos. 8,067,026 and 8,053,000, the complete contents of which are hereby incorporated by reference in entirety. Examples of suitable drugs which may be delivered by a crosslinked electrospun scaffold of the present disclosure include, but are not limited to, antimicrobial agents, protein and peptide preparations (e.g., cytokines), lipids, growth factors (e.g., TGF-β), tissue inhibitor of metalloproteinases (TIMPs), antipyretics, antiphlogistic and analgesic agents, anti-inflammatory agents, vasodilators, antihypertensive and antiarrhythmic agents, hypotensive agents, antitussive agents, antineoplastic agents, local anesthetics, hormone preparations, antiasthmatic and antiallergic agents, antihistaminics, anticoagulants, antispasmodics, cerebral circulation and metabolism improvers, antidepressant and antianxiety agents, vitamin preparations such as vitamin D preparations, hypoglycemic agents, antiulcer agents, hypnotics, antibiotics, antifungal agents, sedative agents, bronchodilator agents, antiviral agents, dysuric agents, antiepileptic drugs, glycosaminoglycans, carbohydrates, nucleic acids, inorganic and organic biologically active compounds, combinations thereof, and the like. Specific biologically active agents include, but are not limited to, enzymes, angiogenic agents, anti-angiogenic agents, antiproliferative agents, growth factors, antibodies, neurotransmitters, psychoactive drugs, anticancer drugs, central nervous system (CNS) therapeutics, antimicrobial agents including antibiotics such as rifampin, chemotherapeutic drugs, drugs affecting reproductive organs, genes, oligonucleotides, combinations thereof, and the like.

Exemplary non-genetic therapeutic agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (o) agents that interfere with endogenous vasoactive mechanisms; (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) beta-blockers, (u) bARKct inhibitors, (v) phospholamban inhibitors, and (w) Serca 2 gene/protein, and the like, including paclitaxel, sirolimus, everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, and Serca 2 gene/protein among others.

Exemplary genetic therapeutic agents for use in connection with the present invention include anti-sense DNA and RNA as well as DNA coding for the various proteins (as well as the proteins themselves): (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor alpha, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

In some embodiments, the active agent is delivered by a vector that is delivered by the crosslinked electrospun scaffold. Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, multiparticles, nanoparticles, or microparticles, with and without targeting sequences such as the protein transduction domain (PTD).

In other embodiments, the active agent that is delivered is or is delivered by cells that are incorporated into the crosslinked electrospun scaffolds of the invention. Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, e.g. to deliver proteins of interest.

In some embodiments, the agent that is delivered is an agent for a vascular treatment regimen, for example, an agent that targets restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and beta-cyclodextrin tetradecasulfate, thrombin inhibitors such as hirudin, hirulog, PPACK (D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α. pathway agents such as thalidomide and analogs thereof, thromboxane A2 (TXA2) pathway modulators such as sulotroban, TIMPs, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Other beneficial agents that may be incorporated into the crosslinked electrospun scaffolds described herein include various metals, (e.g. Ag, Au, etc.); minerals or inorganic agents (e.g. calcium, phosphorous, hydroxyapatite, etc.); agents that release beneficial gases upon contact with an aqueous environment (e.g. agents that release H₂S, etc.); and ocular drugs (e.g., brimonidine, timolol, etc). Furthermore, the agents may include multiparticles, microparticles and nanoparticles coupled to or complexed with or containing any of the agents described herein, or modified with targeting sequence against specific receptors such as epidermal growth factor receptor (e.g., EGF, cetuximab, etc), transferrin receptor (e.g., OX26, etc), folate receptor (e.g., folate acid), etc.

The crosslinked electrospun scaffolds of the invention may be used in any of a number of ways, many of which are known and described in the art. In some embodiments, the crosslinked electrospun scaffolds of the invention may be used to provide support, e.g. as a scaffolding for cell growth in vitro or in vivo, or to hold biological cells, tissues, organs, orifices, etc. in a desired position or shape. In this embodiment, the electrospun matrices have a role that is largely structural, e.g. by functioning as a stent to open an artery, or as a support for the growth and shaping of artificial organs in vitro, etc. In these embodiments, the crosslinked scaffolds of the invention may or may not further comprise an active agent.

In other embodiments, the crosslinked electrospun materials of the invention are used as vehicles for the delivery of biologically active agents (i.e. agents that are physiologically active) to a targeted site of action in a biological system. In these embodiments, the crosslinked scaffolds may or may not also serve a support function as described above. The biological system may be in vitro (e.g. a cell culture system, in an ex vivo tissue or organ, etc.) or in vivo (e.g. within a living organism). In some embodiments, the living organism is a mammal, e.g. a human, although this need not always be the case. The invention is also intended to encompass, e.g. veterinary applications, as well as extensions to other types of living organisms, including plants (e.g. for the delivery of nutrients, pesticides, etc.). The crosslinked electrospun materials may be used as mucoadhesive patches that can adhere to mucosal membrane such as buccal mucosa and deliver beneficial agents locally or systemically by crossing the buccal mucosa. Other sites include vaginal, rectal, nasal, ocular mucosa, etc. Beneficial agents delivered in this manner include but are not limited to those used for the treatment of local disorders, including motility dysfunction and fungal infections, and agents intended for systemic delivery. The treatment of e.g. reflux can be undertaken in this manner, as can delivery therapeutic agents to damaged mucosa. One or more chemical enhancers or chemical enhancement techniques, or combinations thereof, may be added to or used in conjunction with the electrospun scaffolds described herein.

In some embodiments, a variety of drugs or active agents are delivered via the oral mucosal route using the crosslinked electrospun scaffolds of the invention. These include but are not limited to the exemplary drugs: analgesics such as fentanyl citrate, buprenorphine HCl, buprenorphine HCl, naloxone HCl, proclorperazine, testosterone, nitroglycerine, glyceryl trinitrate, zolpidem, nicotine, miconazole, cannabis-derived agents, sedatives such as: midazolam, triazolam and etomidate, cardiovascular drugs such as captopril, verapamil and propafenone, and insulin. In addition, various vaccine formulations may be delivered by the crosslinked electrospun scaffolds described herein, via oral buccal or other routes.

In some embodiments, the crosslinked electrospun scaffolds of the invention are used as wound dressings or bandages. In these embodiments, the electrospun scaffolds provide protection for the wound during healing, and/or may provide structural support for cells or tissues, and/or may include at least one therapeutic agent that is delivered to the wound site via the electrospun material. The wounds that are so treated may be external wounds (e.g. cuts, abrasions, etc. to the skin) or internal wounds (e.g. those caused by purposeful surgical procedures, or puncture wounds, etc.). Those of skill in the art will recognize that for such applications, the crosslinked electrospun matrix may be formed into any suitable size, e.g. as a flat sheet, as a cylinder, a disc, etc. which can be applied to the wound. In some embodiments, electrospun scaffolds may be crosslinked in situ upon exposure to UV light in the presence of acrylate-containing compounds.

The crosslinked electrospun scaffolds of the invention may serve as implantable devices. For example, they may function as nerve guides (e.g. for the repair of severed nerves), or as stents for use in cardiovascular surgery, or fabricated into wafer containing chemotherapeutics (e.g. anticancer drugs, nucleic acids, etc) and implanted into the brain for brain tumor treatment.

The crosslinked scaffolds of the invention are well suited to the delivery of therapeutic and/or biologically active agents by the oral-buccal route. The scaffolds can be formulated using particularly biocompatible substances such as gelatin, and crosslinked with acrylates to provide a scaffold with enhanced durability, compared to uncrosslinked gelatin scaffolds. As with the other embodiments described herein, the extent of crosslinking can be adjusted to achieve any desired level commensurate with the desired application, e.g. rapid dissolution and release of active agents; long term, sustained release of agents; etc.

The invention also encompasses methods of making the crosslinked, electrospun materials of the invention. The methods generally involve electrospinning a suitable solution to form an electrospun scaffold, associating a photoreactive acrylate with the scaffold, and then exposing the scaffold with the associated photoreactive acrylate to a source of radiation that is suitable for activating the acrylate, for example, ultraviolet (UV) light. The acrylate species may be associated with the scaffold in any suitable manner, e.g. by soaking the scaffold in a solution of acrylate, allowing the acrylate solution to “wick” into the scaffold, by spraying or otherwise coating or permeating the scaffold with acrylate solution, etc. In some embodiments, the entire scaffold is contacted with a photoactivatable acrylate, although this need not always be the case, as the acrylate may be differentially applied in order to form regions of crosslinking and regions which are not crosslinked. Further, more than one type of acrylate may be used, or different types or concentrations of acrylates may be used on different section of the scaffold, e.g. to form gradients of crosslinking (and hence of permeability), or to form regions with different properties, e.g. different rates of degradation, or containing different active agents, etc.

Those of skill in the art are familiar with photoactivation of acrylates, e.g. in the context of forming hydrogels. Generally, polymerization of acrylate species is photo-initiated by irradiation with UV light, resulting in photolysis of the acrylate to produce free radicals. Polymerization then proceeds via free radical polymerization, in which acrylate free radicals react with each other to form polymers, and also with other molecules in the environment such as scaffolding components. Reaction with a scaffolding component results in chain termination, but also in the linking of the polymer chain to the scaffolding. In this manner, relatively random crosslinking of scaffolding components and polymers occurs, thereby forming an interconnected “mesh” structure within the scaffold and increasing the rigidity and/or tensile strength of the scaffold. Crosslinking also increases the effective interior solid surface area of the scaffold, permitting higher loading of the scaffold with molecules of interest.

Incorporation of bioactive agents into the scaffold may be accomplished by adding or inserting the agents (e.g. suspended or dissolved in a suitable solution) into the scaffold at some point before crosslinking is carried out. For example, the agents may be added before the acrylate, or may be added to the acrylate solution, or may be added after the scaffold is permeated with acrylate solution. The scaffolding is then crosslinked and the agent is trapped inside, or at least egress of the agent from the scaffolding is slowed. However, embodiments in which the agents are added after crosslinking are also encompassed by the invention.

The crosslinked scaffolds of the invention, due to the high level of durability and high loading capacity, are ideal for use in applications which require long term, sustained release of active agents. The crosslinking of the scaffolds can be tuned or tailored so as to achieve any desired degree of resistance to degradation, so that the scaffolds may remain largely intact e.g. for days, weeks, months, or even longer.

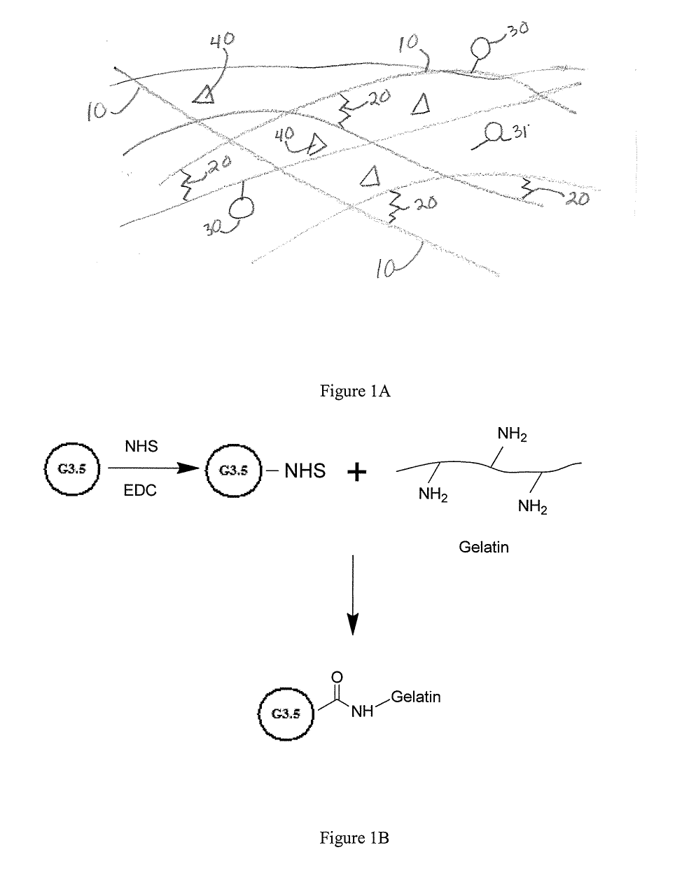

FIG. 1A depicts a schematic representation of the interior of a crosslinked electrospun scaffold or matrix as described herein. Shown are electrospun fibers 10 connected by crosslinks 20, with (optional) dendrimers 30 connected (e.g. covalently linked) to the fibers and (optional) dendrimers 31 present in and amongst the fibers e.g. by being sterically trapped. Optional active agents 40 (e.g. a drug, a metal such as silver, a nanoparticle, etc.) are also present within the scaffolding.

The foregoing examples serve to further illustrate various embodiments of the invention but should not be construed so as to limit the invention in any way.

### EXAMPLES

### Example 1

Electrospun Gelatin/Dendrimer Scaffold with Silver

Electrospinning is a popular technique used for the fabrication of nanoscale structures for various applications like wound dressings, drug delivery vehicles and tissue engineered scaffolds (Huang et al. 2004). The scaffolds produced from natural, biodegradable polymers have very small fiber diameter ranging from nano to micrometers which is suitable to replicate the structural morphology of the natural extracellular matrix of native tissues and organs (Huang et al. 2004).

In this study, gelatin was the major component used since it is a natural biopolymer derived from collagen. It is biocompatible, biodegradable and can be commercially available at a relatively low cost (Zhang et al. 2005). It is popularly used in the field of medicine as a sealant for vascular prosthesis and as a wound dressing. However, gelatin is easily soluble in water and electrospun gelatin fibers can easily lose their structural stability in an aqueous medium. Hence, gelatin-based scaffolds need to be crosslinked or incorporated with stabilizing polymers to retain its mechanical integrity as a tissue engineered construct (Zhang et al. 2005). Dendrimer can be covalently bound to gelatin and electrospun into a scaffold for drug encapsulation and drug delivery.

Silver was selected as an antimicrobial agent due to its broad range of antimicrobial activity against gram-positive and gram-negative bacteria (Hromadka et al. 2008). It can also inhibit antibiotic-resistant bacteria like methicillin-resistant Staphylococcus aureus (MRSA) and vancomycin-resistant enterococci (VRE) when used at proper concentrations (Warriner and Burrell 2005). Silver can kill micro-organisms by multiple mechanisms, it can change the structure and function of a bacterial cell by altering its protein structure or rupture the bacterial cell wall or block the respiratory pathway (Warriner and Burrell 2005). Silver-based wound dressings and creams are used for wound healing and to maintain a microbe free environment at the wound site (Warriner and Burrell 2005). Silver-based dressings are particularly used in burn wounds, chronic leg ulcers, diabetic wounds and traumatic injuries (Ip et al. 2006). There are various ways by which silver can be incorporated in the dressing such as silver nitrate, silver sulfadiazine, silver calcium phosphate or in the form of nanocrystalline silver (Warriner and Burrell 2005). The silver can be released from the dressing by means of diffusion to the surface of the wound (Agarwal et al. 2009).

Apart from the advantages of including silver in a dressing, there are certain difficulties faced with current topical silver dressings such as slow rate of release, staining at the wound area, rapid consumption of silver ions and patient comfort (Warriner and Burrell 2005). Due to these issues, silver based dressings need to be investigated and an optimum concentration of silver introduced into the dressing needs to be selected.

### Experimental Methods

### Preparation of Gelatin-Dendrimer Conjugates

The protocol used for the preparation of gelatin-dendrimer conjugate was a slight modification to that used by Alicia Smith Freshwater (Smith-Freshwater 2009). Gelatin was conjugated with half-generation PAMAM dendrimer G3.5. Briefly, 120 μl of G3.5 in methanol stock solution was dried by rotary evaporation and re-dissolved in 2 ml of distilled water. This solution was vortexed thoroughly and mixed with 3 mg of NHS and 5 mg of EDC while stirring for 24 h to achieve surface activated G3.5 (i.e., G3.5-NHS). To prepare the gelatin solution, 20 mg of gelatin was added to 20 ml of 0.1N NaHCO₃ solution and completely dissolved by stirring at 80° C. until it formed a clear solution. At 24 h, the gelatin solution was added to G3.5-NHS solution and kept in an ice bath for 4 h. It was then centrifuged for 20 min at 10 rpm and the supernatant was added drop wise to 50 ml of ethyl ether and refrigerated for 24 h. It was then centrifuged for 20 min at 10 rpm and the precipitate was collected. The precipitate was further purified by rapid dialysis using 12-14 kDa MWCO dialysis tubing. The purified solution was lyophilized by FTS to obtain gelatin-dendrimer conjugates.

### Preparation of Sample Solutions for Electrospinning

Eight different sample solutions were prepared using HFP as a solvent. The types of scaffolds electrospun and their constituents are summarized in Table 1. The reason for electrospinning eight different scaffolds with varying amounts of silver was to compare the characteristics of these scaffolds against each other and to determine the optimum silver composition against infection. The solutions were mixed thoroughly for 24 h on a shaker plate prior to electrospinning.

### Electrospinning

In the electrospinning process, electrical charge is applied to draw fine fibers from the solution. The solution for electrospinning is loaded into a syringe and a positively charged electrode is attached to the needle of the syringe. The voltage applied results in an electric field and the drop of polymer solution at the tip of the needle is altered into a conical shape known as the Taylor cone. As the strength of the electric field increases, the polymer solution jet is elongated to form long, thin fibers as a result of solvent evaporation. The fibers are collected on a collector or mandrel that is grounded. The mandrel undergoes translation and rotation for the uniform deposition of the scaffold.

TABLE 1
Solutions prepared for electrospinning scaffolds
(The solutions were prepared in 10 ml of HFP)

| | Gelatin | Gelatin-dendrimer | Silver acetate |
|---|---|---|---|
| Scaffold | (mg/ml) | conjugate (mg/ml) | (mg/ml) |
| S1 | 100 | 0 | 0 |
| S2 | 100 | 0 | 3.3 |
| S3 | 100 | 0 | 1.65 |
| S4 | 100 | 0 | 0.825 |
| S5 | 96 | 4 | 0 |
| S6 | 96 | 4 | 3.3 |
| S7 | 96 | 4 | 1.65 |
| S8 | 96 | 4 | 0.825 |

Particularly, the electrospinning solution was loaded into a 10 ml Becton Dickinson syringe and placed in a KD Scientific syringe pump for electrospinning. The syringe pump was set to deliver the solution at a rate of 5 ml/h. A voltage of 25 kV was applied to the needle of the syringe by a high voltage power supply (Spellman CZE1000R, Spellman High Voltage Electronics Corporation). The mandrel chosen for collecting the fibers was a flat, stainless steel mandrel 7.5 cm×2.5 cm×0.5 cm (L×W×T). It was placed approximately 125 mm from the needle tip and rotated at ˜500 rpm for uniform collection of the fibers. After electrospinning was completed, the scaffold was carefully removed from the mandrel, placed in a fume hood for degassing and stored in a moisture-free environment.

### Crosslinking

After electrospinning, the scaffolds were crosslinked to increase structure stability and mechanical properties. For each scaffold, 100 μl of PEG diacrylate, 4 mg of dimethoxyphenylacetophenone (photo-initiator) and 2 ml of ethanol were used to prepare the crosslinking solution. The solution was poured onto a scaffold of 7.5 cm×5 cm and of varied thickness and allowed to stay for about 30 min. The scaffold was then held under UV light for 2 min on each side. This method is referred to as the solution method. As an alternative method, vapors were used for crosslinking the scaffolds. The solution was heated in a water bath and the scaffold was crosslinked by the vapors. It was then held under UV light for 2 min on each side. This method is referred to as the vapor method. The scaffolds crosslinked by the vapor method did not retain their structure in aqueous medium and could be only characterized for morphology, fiber diameter, and tensile properties.

### Characterization

### Ninhydrin Assay

Ninhydrin assay was performed to confirm the conjugation of dendrimer to gelatin. The ninhydrin stock solution was prepared by dissolving 30 mg of ninhydrin in 10 ml of ethanol. Five different concentrations of gelatin were prepared and mixed with 1 ml ninhydrin solution and a standard curve was obtained using UV-Vis spectrophotometer. 1 mg of G3.5-gelatin conjugate was mixed with 1 ml of DI water and 1 ml of ninhydrin solution. This mixture was heated to approximately 80° C. for 5-10 min and cooled to 20-25° C. and the absorbance was measured at 570 nm. The absorbance value of G3.5-gelatin conjugate mixed with ninhydrin was compared to the standard curve of gelatin mixed with ninhydrin.

### Scanning Electron Microscopy (SEM)

SEM images of the scaffolds were taken to characterize the structure and morphology of the scaffolds. A small piece of sample from each scaffold was cut and gold sputter coated. Images were taken by Scanning Electron Microscope Model 550 at a magnification of 1200×. A scale bar of 10 μm is presented on each figure. Fiber diameter was calculated by using the UTHSCSA Image tool Version 3.0 to measure 30 randomly chosen fibers.

### Tensile Testing

Tensile studies of the scaffold were performed to analyze the mechanical properties of the scaffolds. Tensile studies were done on the MTS Bionix 200® Mechanical testing system with a 100 N load cell. Six dog-bone shaped samples were cut out from each scaffold using a punch die. The thickness of the samples was measured in inches and the scaffolds were placed in the metal grips of the mechanical testing system moving at a rate of 10 mm/min. Stress, strain, modulus and energy to break were measured by the MTS Testworks software (version 4.04A).

### Porosity Measurements

Scaffold porosity was measured by taking out 1 cm×1 cm samples from the scaffold and measuring the mass in g and thickness which ranged from 0.032 cm to 0.33 cm. Porosity was calculated by the formula:

$$P = [ 1 - \frac{{V}_{g}}{{V}_{a}} ] \times 100$$

Where, V_{g}=Mass of scaffold/Density of collagen (1.41 g/cm³)

Vₐ=Apparent volume of the square section 1 cm×1 cm×thickness Three samples (n=3) were used from each type of scaffold for the porosity measurements.

### Permeability and Pore Size Measurements

Permeability was measured by an apparatus designed by Scott Sell (Sell et al. 2008). 12 mm discs were punched out from the scaffolds and the time taken for 10 ml of water to pass through the disc was noted.

Permeability was calculated as (Can and Hardin 1987):

$$τ = \frac{Q μ T}{tAP}$$

### Where,

τ=Permeability measured in Darcy
Q=volume flowing through the system
T=scaffold thickness
μ=fluid viscosity (0.89 cP)
t=time taken for 10 ml water to flow through the scaffold (in seconds)
A=cross sectional area of scaffold (πr²)
P=applied pressure (ρgh) in atm
Where, ρ=density of water (1000 kg/m³)
   g=gravitational force (9.8 m/s²)
   h=total height of the system (m)
      The average pore size was calculated as (Can and Hardin 1987):

$$r = \frac{0.5093}{{τ}^{- 1 \text{/} 2}}$$

Three samples (n=3) were used from each type of scaffold for permeability measurements.

### Adsorption and Swelling Studies

Simulated wound fluid (Parsons et al. 2005) (SWF) was used as the medium for swelling studies and antimicrobial activity to mimic the clinical conditions. SWF consists of 50% calf serum and 50% maximum recovery diluent (0.1% w/v peptone and 0.9% w/v sodium chloride) (Parsons et al. 2005). 2.5 cm×2.5 cm of samples were cut out from each of the scaffolds and weighed (W_{d}). They were immersed in 5 ml of SWF at room temperature. The samples were taken out of the fluid, blot dried and weighed (Wₛ) at 10 min, 20 min, 30 min, 60 min, 90 min, 120 min, 24 h and 48 h. The swelling ratio (%) was calculated by the formula (Parsons et al. 2005):

$$Swelling ratio \% = [ \frac{{W}_{s} - {W}_{d}}{{W}_{d}} ] \times 100$$

Three samples (n=3) were used from each type of scaffold for swelling studies.

### In Vitro Degradation Studies

The in vitro degradability of the scaffolds without silver (S1 and S5) was evaluated. Four different media and conditions were used for the degradation studies: (i) incubation in Dulbecco's modified Eagle's medium (DMEM) supplemented with 10% fetal bovine serum (FBS) at 37° C., (ii) DMEM supplemented with 10% FBS at room temperature, (iii) incubation in SWF at 37° C. and (iv) incubation in cell conditioned medium at 37° C. (Cell conditioned medium was DMEM supplemented with 10% FBS used for culturing confluent BJ-hTERT fibroblasts for 24 h in 96×16 mm sterile petri dish). Nine samples from each scaffold of size 1 cm×1 cm were weighed and immersed in 1.5 ml of each of the media mentioned above, for 24 h. After 6 h, 12 h and 24 h three samples of each scaffold type were taken out from all media, centrifuged for 20 min, frozen, then lyophilized and weighed. The average ratio of weight loss due to the degradation in each scaffold was calculated using the formula:

$$Ratio of weight loss ( \% ) = [ \frac{{W}_{o} - {W}_{d}}{{W}_{o}} ] \times 100$$

Where, Wₒ=initial weight

W_{d}=weight of the sample after degradation

### Antimicrobial Activity of Silver

The antimicrobial activity of silver was tested against common wound pathogens-gram positive Staphylococcus aureus (strain N315) and gram negative Pseudomonas aeruginosa (strain PA01). Colony plates of Staphylococcus aureus and Pseudomonas aeruginosa were cultured from the respective bacterial strains. 1 L of Luria agar was prepared containing 10 g of Tryptone, 5 g of yeast extract, 10 g of NaCl and agar to a final concentration of 1.5%. All the components were dissolved in 1 L of DI water. The medium was autoclaved at 121° C. for 15 to 20 min and poured onto sterile petri plates and allowed to dry. Bacterial culture was inoculated using 1 colony in 3-4 ml of SWF and incubated at 37° C. overnight. 10 fold serial dilutions of the pure bacterial culture were made in SWF and 10⁵ dilution repeats were prepared in test tubes. 2.5 cm×2.5 cm sample taken out from each scaffold were inserted into the 10⁵ dilution test tubes and incubated at 37° C. One test tube containing no scaffold was used as control. 100 μl (0.1 ml) of aliquot was taken out from each of the test tubes at 4 h, 24 h and 48 h and plated on luria agar plates. The plates were incubated at 37° C. in the incubator overnight and observed for any bacterial growth thereafter. After incubation, the number of colonies present was counted and colony forming units/ml (cfu/ml) was reported.

### Silver Release Studies

The silver release from the scaffolds was studied in PBS. 2.5 cm×2.5 cm of samples were taken out from the scaffolds, weighed and immersed into a capped glass vial containing 20 ml (0.02 L) of PBS. The glass vial was kept on a stir plate and the temperature was maintained at 37° C. At pre-determined time points: 1 h, 2 h, 3 h, 4 h, 24 h, 48 h, 72 h, 96 h, 120 h, 144 h, 168 h, 192 h, 216 h, 240 h and 264 h, 10 ml (0.01 L) of PBS was transferred to a capped tube for silver content analysis. 10 ml of fresh PBS was added to the vial to maintain the volume of the medium for continuous observation. Silver content was analyzed by Inductively Coupled Plasma-Optical Emission Spectroscopy (ICP-OES/ICP Varian Vista MPX). ICP-OES is a technique used for analysis of trace metals present in a sample. The presence of the metals is detected by electromagnetic radiation emitted by excited atoms or ions, at a wavelength characteristic to a particular metal (Mermet 2005; Stefánsson et al. 2007). The concentration of the metal can be calculated by the intensity of the emission.

Different concentrations of aqueous silver standards were prepared from a stock solution of 1000 ppm (mg/L) silver standard. The intensity values of the known concentration of silver standards and the aliquots were recorded by ICP-OES. The calibration curve of silver was used as a reference to calculate the concentration of silver in each of the aliquots. The concentration of silver in each aliquot ([concentration]ₜₒ) was obtained in the units of parts per billion (ppb or μg/L). The amount released at each time point was calculated as follows:

$$\% Cumulative amount released ( t ) = [ \frac{{[ concentration ]}_{t} \times 0.02 L + {[ concentration ]}_{to} \times 0.01 L}{Initial amount of silver in the sample ( μ g )} ] \times 100$$

### Statistical Analysis

Statistical analysis was performed on mean scaffold fiber diameters, tensile testing values, porosity, permeability, pore size, swelling ratio (%) and degradation. All statistical analysis was based on one way analysis of variance (ANOVA) and Tukey's test for significance, performed on Minitab statistical software. A p-value less than 0.05 was considered statistically significant. Graphical representations of mean data were constructed with Microsoft Excel 2007 with error bars representing standard deviations.

### Preparation and Characterization of Gelatin-Dendrimer Conjugates

Coupling of G3.5 to gelatin was based on 1-Ethyl-3-[3-dimethylaminopropyl]carbodiimidehydrochloride/N-hydroxysuccinimide (EDC/NHS) chemistry and the reaction mechanism is shown in FIG. 1. EDC along with NHS activated the terminal carboxyl groups of G3.5. The carboxyl groups and amine groups form an amide bond, hence coupling dendrimer to the gelatin backbone.

The conjugation of gelatin and G3.5 was confirmed by the ninhydrin assay. Ninhydrin produces a chromophore when mixed with amine containing compounds and its presence can be detected by measuring the absorbance value by UV-Vis spectrophotometer at a wavelength of 570 nm. 1 mg/ml of pure gelatin mixed with 1 ml of ninhydrin was found to have an absorbance value of 0.009 whereas 1 mg/ml of G3.5-gelatin mixed with 1 ml of ninhydrin solution gave an absorbance value of 0.002. The reduced absorbance was attributed to the decrease in the number of free amine groups and hence confirmed the conjugation of dendrimer to gelatin.

### Morphology, Fiber Diameter and Mechanical Properties

Electrospinning technique for fabrication of fiber scaffolds is gaining popularity due to its simplicity and ease of use (Kumbar et al. 2008). Electrospun scaffolds exhibit similarity in morphology to natural extra-cellular matrix (ECM), which is beneficial for tissue growth. Electrospinning can produce randomly oriented or aligned, continuous fibers which have high porosity and high surface area (Sill and von Recum 2008).

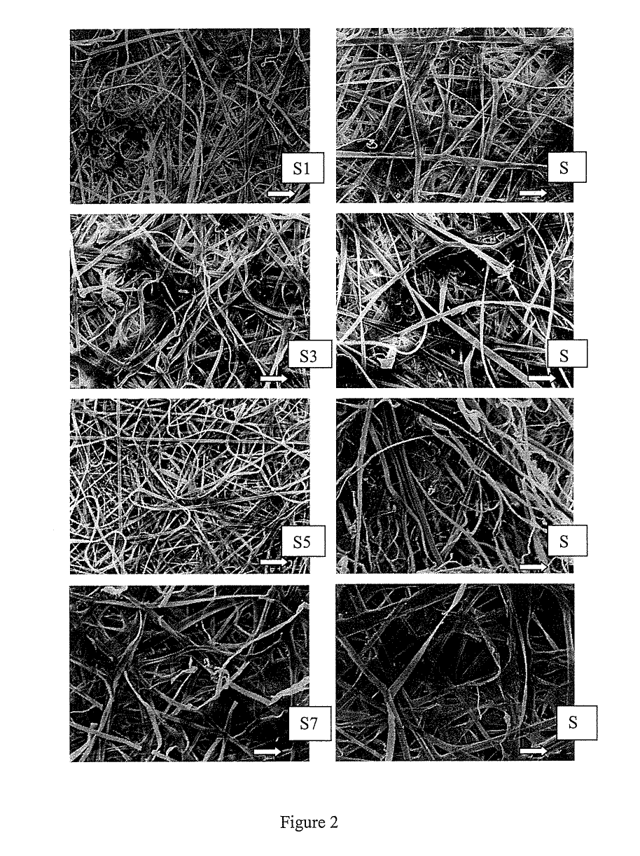

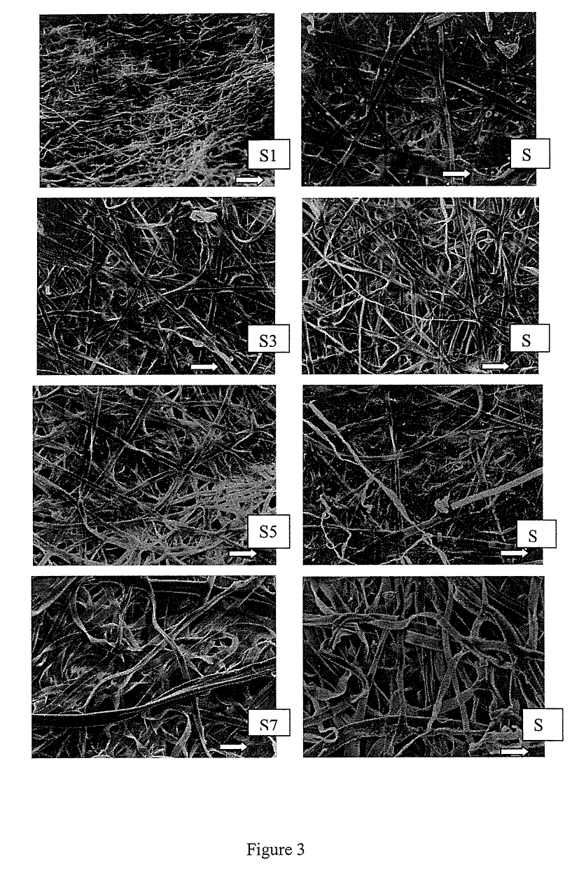

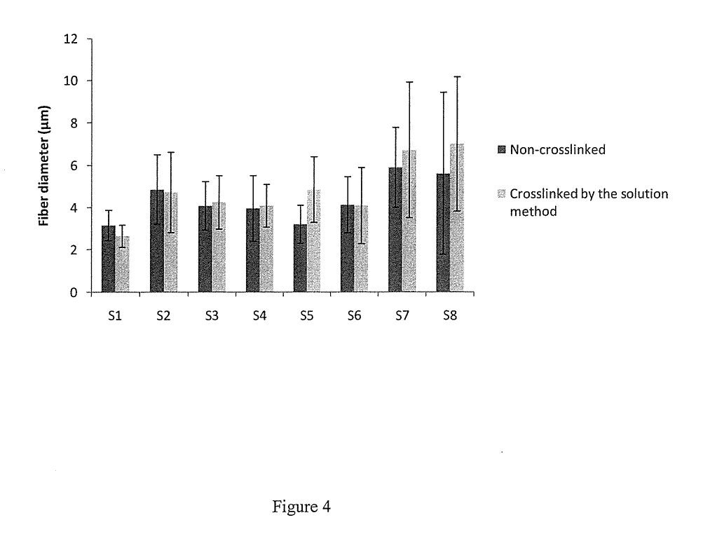

Pure gelatin alone or with gelatin-dendrimer conjugates was electrospun to form fiber scaffolds. The scaffolds were further crosslinked to improve their structural stability. The data shown and discussed in this example are based on non-crosslinked scaffolds and scaffolds crosslinked by the solution method. The structure, morphology, mechanical properties and swelling ability of the scaffolds were evaluated. Different concentrations of silver acetate were incorporated in the scaffolds to determine the antimicrobial efficacy and silver release kinetics. SEM images of non-crosslinked scaffolds and scaffolds crosslinked by the solution method are shown in FIG. 2 and FIG. 3, respectively. It is observed that the scaffolds crosslinked by the solution method retained the fiber structure. There are many factors that affect the fiber diameter and morphology, such as concentration of polymer, viscosity, voltage applied, diameter of the needle and the rate at which the polymer solution is delivered (Lee et al. 2008). The fiber diameter of the non-crosslinked scaffolds ranged from 3.15 to 5.88 μm and that of the scaffolds crosslinked by the solution method ranged from 2.64 to 6.98 μm. A graphical representation of the data is shown in FIG. 4. Also, statistical differences were analyzed using ANOVA in Minitab statistical software. It was observed that for both non-crosslinked and crosslinked scaffolds by the solution method, the fiber diameter of the gelatin-dendrimer scaffolds (S5, S6, S7, S8) was larger than that of the scaffolds containing gelatin only (S1, S2, S3, S4). Also it was observed that as the silver concentration in the gelatin scaffolds decreased, the fiber diameter decreased but for the gelatin-dendrimer scaffolds as the silver concentration in the gelatin scaffolds increased. Half generation PAMAM dendrimers have a negative charge which could also have an influence on the electric field during electrospinning, thereby affecting the fiber diameter.

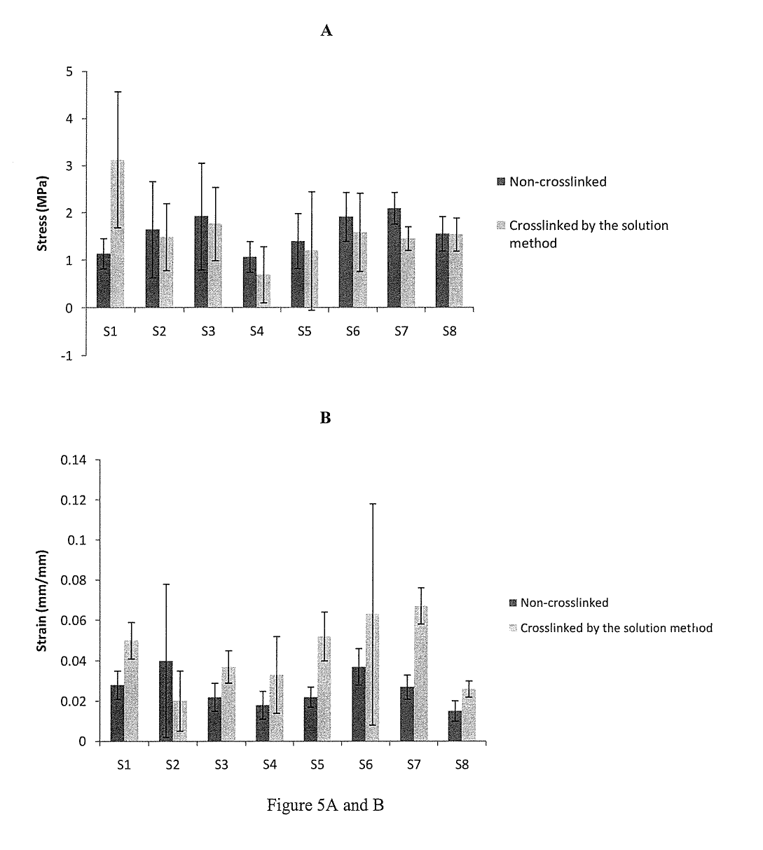

Mechanical properties of a scaffold need to be evaluated for durability, stress resistance, flexibility and elasticity (Boateng et al. 2008). Peak stress, strain at break, energy to break and modulus of all scaffolds were determined by MTS Bionix and Testworks4.0 software. Tensile strength is the maximum stress a scaffold can withstand before breaking and determines the hardness of the scaffold. It also depends on the type and amount of polymer in the scaffold (Boateng et al. 2008). Strain at break describes the ductility and brittleness of the scaffold and also tells about the elongation of the scaffold at breaking point (Boateng et al. 2008). The mechanical properties and fiber diameter of the non-crosslinked and crosslinked scaffolds by the solution method are shown in Table 2 and Table 3, respectively; and a graphical representation of stress, strain and modulus data is shown in FIGS. 5A-C. The mean stress of the non-crosslinked scaffolds ranged from 1.063 to 2.087 MPa and that of crosslinked scaffolds by the solution method ranged from 0.692 to 3.125 MPa with a pooled standard deviation of 0.8616. Mean stress for crosslinked scaffolds by the solution method is statistically higher in the S1 scaffold type which contained only gelatin. It was observed that the stress values were higher for the non-crosslinked scaffolds as compared to the crosslinked ones except for scaffold S1. This behaviour was also observed in the modulus results. The scaffolds containing silver displayed higher stress values than scaffolds without silver. The mean strain for the noncrosslinked scaffolds ranged from 0.015 to 0.040 mm/mm and for the crosslinked scaffolds by the solution method ranged from 0.020 to 0.067 mm/mm. The strain values for crosslinked scaffolds were higher than the non-crosslinked scaffolds except for scaffold S2.

Further characterization to determine the porosity, permeability, swelling ratio, antimicrobial activity and silver release kinetics was performed only on scaffolds crosslinked by the solution method since they could retain their stability in aqueous medium.

TABLE 2
Tensile studies and fiber diameter of the non-crosslinked scaffolds

| | Fiber | | | Strain at | |
|---|---|---|---|---|---|
| | diameter | Peak stress | | break | Energy to |
| Scaffold | (μm) | (MPa) | Modulus (MPa) | (mm/mm) | break (N * mm) |
| S1 | 3.15 ± 0.72 | 1.136 ± 0.319 | 47.134 ± 6.463 | 0.028 ± 0.007 | 0.203 ± 0.097 |
| S2 | 4.85 ± 1.64 | 1.646 ± 1.016 | 93.636 ± 46.672 | 0.040 ± 0.038 | 0.272 ± 0.286 |
| S3 | 4.07 ± 1.15 | 1.922 ± 1.128 | 99.276 ± 45.636 | 0.022 ± 0.007 | 0.13 ± 0.050 |
| S4 | 3.95 ± 1.96 | 1.063 ± 0.323 | 64.841 ± 15.302 | 0.018 ± 0.007 | 0.107 ± 0.064 |
| S5 | 3.20 ± 0.90 | 1.399 ± 0.578 | 80.525 ± 20.326 | 0.022 ± 0.005 | 0.183 ± 0.090 |
| S6 | 4.12 ± 1.34 | 1.908 ± 0.519 | 66.580 ± 18.809 | 0.037 ± 0.009 | 0.476 ± 0.173 |
| S7 | 5.88 ± 1.88 | 2.087 ± 0.335 | 104.835 ± 18.741 | 0.027 ± 0.006 | 0.332 ± 0.174 |
| S8 | 5.59 ± 3.83 | 1.549 ± 0.363 | 100.319 ± 27.641 | 0.015 ± 0.005 | 0.137 ± 0.079 |

TABLE 3
Tensile studies and fiber diameter of the scaffolds crosslinked by the solution method

| | Fiber | | | Strain at | |
|---|---|---|---|---|---|
| | diameter | Peak stress | | break | Energy to |
| Scaffold | (μm) | (MPa) | Modulus (MPa) | (mm/mm) | break (N * mm) |
| S1 | 2.64 ± 0.53 | 3.125 ± 1.443 | 80.835 ± 24.326 | 0.050 ± 0.009 | 0.060 ± 0.366 |
| S2 | 4.71 ± 1.90 | 1.485 ± 0.703 | 92.709 ± 35.127 | 0.020 ± 0.015 | 0.011 ± 0.075 |
| S3 | 4.24 ± 1.27 | 1.760 ± 0.774 | 58.330 ± 20.242 | 0.037 ± 0.008 | 0.306 ± 0.205 |
| S4 | 4.08 ± 1.02 | 0.692 ± 0.587 | 38.271 ± 12.854 | 0.033 ± 0.019 | 0.115 ± 0.140 |
| S5 | 4.85 ± 1.55 | 1.196 ± 1.246 | 53.443 ± | 0.052 ± 0.012 | 0.278 ± 0.319 |
| S6 | 4.08 ± 1.80 | 1.581 ± 0.826 | 59.370 ± 27.612 | 0.063 ± 0.055 | 0.422 ± 0.203 |
| S7 | 6.7 ± 3.19 | 1.447 ± 0.250 | 33.804 ± 8.913 | 0.067 ± 0.009 | 0.646 ± 0.102 |
| S8 | 6.98 ± 3.16 | 1.533 ± 0.348 | 69.395 ± 21.298 | 0.026 ± 0.004 | 0.192 ± 0.062 |

### Scaffold Porosity, Permeability, Swelling and Degradation Studies

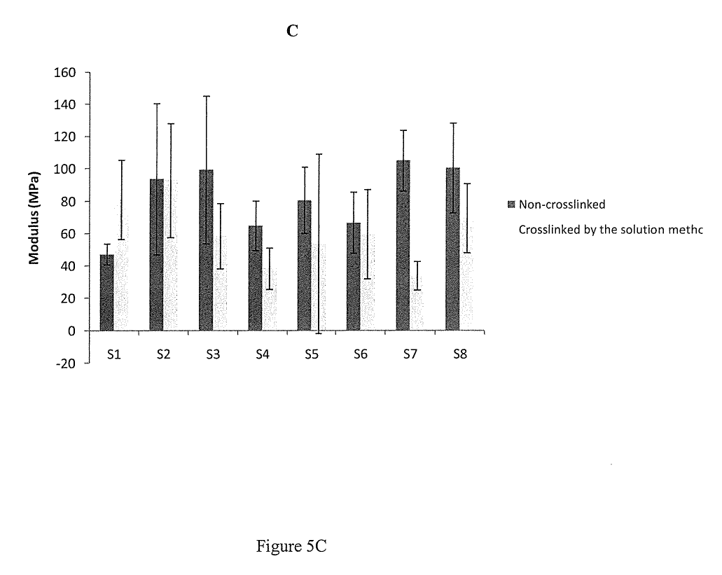

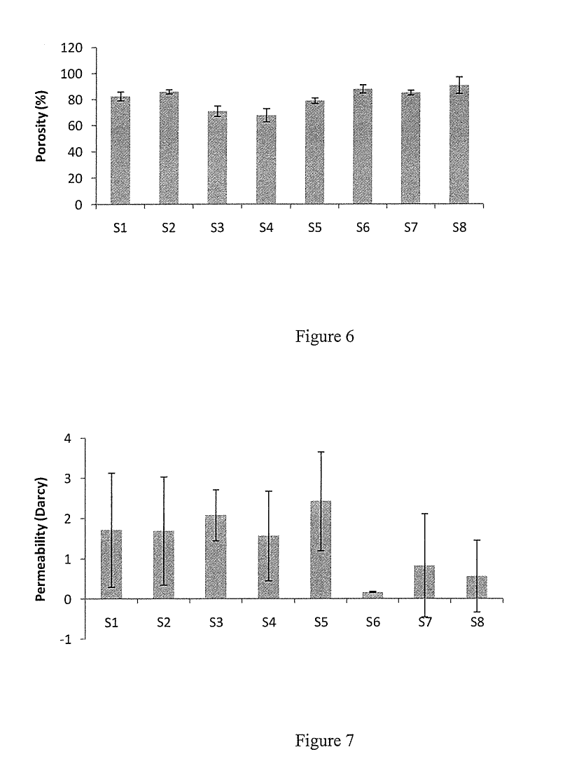

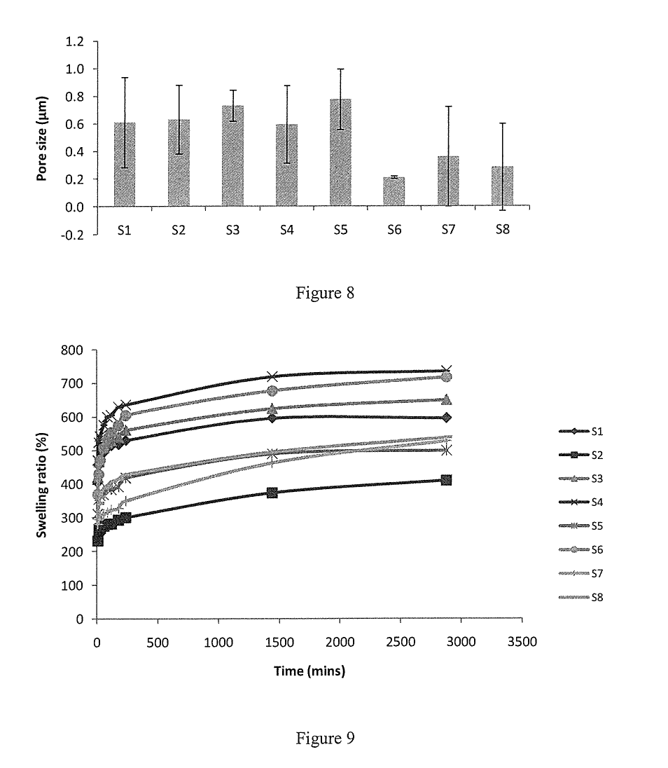

The porosity, permeability and swelling ability of the scaffolds were evaluated to verify their capability for medium exchange. Porosity is the measure of void space within the scaffolds. The graphical data for porosity is shown in FIG. 6. The porosity of the scaffolds crosslinked by the solution method ranged from 67.56 to 90.42% which is suitable as a tissue engineered scaffold for adequate moisture and oxygen exchange to underlying cells (Freed et al. 1994). The porosity of scaffold S8 was significantly higher than the porosity of scaffolds S3, S4 and S5 and highest among all the porosity values. Permeability is the measure of the ease of flow of fluid through the scaffold. Permeability ranged from 0.1673 to 2.428 Darcy and is depicted in FIG. 7. It is observed that the gelatin-dendrimer scaffolds containing silver had lower permeability values as compared to the gelatin scaffolds containing silver but the statistical analysis shows that the data is not statistically significant. This lower permeability values of the gelatin-dendrimer scaffolds containing silver may be due to their higher fiber diameter. Pore size of the scaffolds show a similar trend to what was found in permeability as shown in FIG. 8.

Adsorption and swelling studies were done to determine the swelling capacity of the scaffolds. Wounds that have extensive bleeding and heavy amounts of exudate require dressings to absorb quickly and maintain a clean environment at the wound site. The swelling studies were carried out in the SWF to replicate the clinical conditions. A graphical representation of the data is shown in FIG. 9 and a statistical analysis of the swelling ratio values at 48 h was carried out (not shown). All the scaffolds demonstrated a good swelling and absorbing capacity. The swelling ratio varied from 415 to 626% at the end of 48 h (2880 min). It is observed that scaffold S4 had the highest swelling ratio.

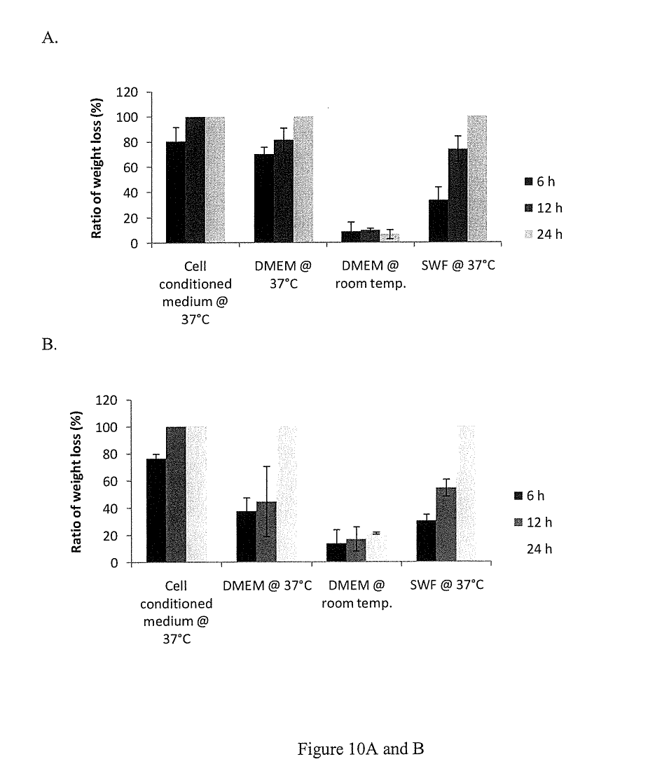

Gelatin is easily soluble and therefore scaffolds containing gelatin need to be evaluated for degradation. The scaffolds without silver (S1 and S5) were evaluated for degradation studies to observe the effect of temperature and other factors, if any, on the rate of degradation of the scaffold. It was observed that temperature did play a role in the rate of degradation as shown in FIGS. 10A and B for scaffold S1 and S5. A statistical analysis of the data was carried out (not shown). For both the scaffolds, the weight loss (%) was significantly lower when tested at room temperature as compared to incubation at 37° C. The type of medium also affected the rate of degradation. Both the scaffolds degraded completely at the end of 24 h in all media at 37° C., with the weight loss being highest during incubation in cell conditioned medium. It was also observed that S5 which contained gelatin-dendrimer conjugate had lesser degradation than S1 which contained only gelatin when incubated in same media at 37° C.

### Antimicrobial Assay

The scaffolds crosslinked by solution method were tested for antimicrobial efficacy against two common wound pathogens, gram positive Staphylococcus aureus and gram negative Pseudomonas aeruginosa. Gram positive bacteria do not contain any outer membrane but have a thick peptidoglycan layer and stain dark blue or violet by Gram's staining. In contrast, gram negative bacteria contain an outer membrane but have a thin peptidoglycan layer and stain pink by Gram's staining. Images of the petri plates were taken (not shown) and the results for colony forming units/ml are shown in Table 4 and Table 5. It is observed that the growth of both bacteria increased in a span of 48 h for the culture test tubes with no sample (control) and those containing scaffold samples without silver (S1 and S5). For Staphylococcus aureus, it was observed that at the end of 4 h, bacterial growth was completely inhibited in the test tubes containing S2, S3, S5 and S6, but there was some growth in the test tubes containing S4 and S8. Overall it was observed that all the scaffold samples containing silver inhibited the growth of Staphylococcus aureus by the end of 48 h. For Pseudomonas aeruginosa, it was observed that all the scaffold samples containing silver inhibited the growth of bacteria by the end of 48 h.

TABLE 4
Antimicrobial activity against Staphylococcus aureus

| Scaffold type | 4 h | 24 h | 48 h |
|---|---|---|---|
| Control | 10 × 10⁷ cfu/ml | 24 × 10⁷ cfu/ml | Bacterial lawn |
| (untreated) | | | |
| S1 | 5.1 × 10⁷ cfu/ml | 6 × 10⁷ cfu/ml | Bacterial lawn |
| S2 | No colonies | No colonies | No colonies |
| S3 | No colonies | No colonies | No colonies |
| S4 | 0.7 × 10⁷ cfu/ml | No colonies | No colonies |
| S5 | 6 × 10⁷ cfu/ml | Bacterial lawn | Bacterial lawn |
| S6 | No colonies | No colonies | No colonies |
| S7 | No colonies | No colonies | No colonies |
| S8 | 1.5 × 10⁷ cfu/ml | No colonies | No colonies |

TABLE 5
Antimicrobial activity against Pseudomonas aeruginosa

| Sample type | 4 h | 24 h | 48 h |
|---|---|---|---|
| Control | Bacterial lawn | Bacterial lawn | Bacterial lawn |
| (untreated) | | | |
| S1 | Bacterial lawn | Bacterial lawn | Bacterial lawn |
| S2 | No colonies | No colonies | No colonies |
| S3 | No colonies | No colonies | No colonies |
| S4 | No colonies | No colonies | No colonies |
| S5 | Bacterial lawn | Bacterial lawn | Bacterial lawn |
| S6 | No colonies | No colonies | No colonies |
| S7 | No colonies | No colonies | No colonies |
| S8 | No colonies | No colonies | No colonies |

### Silver Release Kinetics

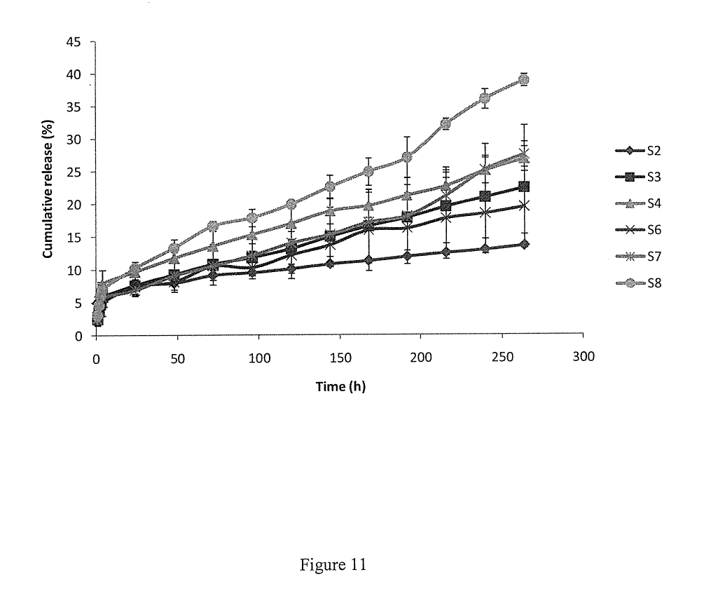

Silver release kinetics were measured by means of diffusion of silver into PBS medium and analyzing the silver content by ICP-OES. A graphical representation of cumulative release of silver (%) over time is shown in FIG. 11. It is observed that all the scaffolds containing silver show a similar drug release pattern over a span of 264 h (short term). Silver release was slow and a very small amount of silver was released at the end of 264 h. It is also observed that larger amount of silver is released from gelatin-dendrimer scaffolds as compared to gelatin scaffolds containing equal amounts of silver (i.e. S2 and S6, S3 and S7, S4 and S8). This may be due to the larger fiber diameter of gelatin-dendrimer scaffolds. Fibers with larger diameter have a greater surface area for diffusion. Comparison of the antimicrobial assay and silver release kinetics revealed that even a low amount of silver released could inhibit any bacterial growth by 48

### CONCLUSIONS

This work demonstrated that silver containing fiber dressings composed of gelatin with or without gelatin-dendrimer conjugates could be fabricated by electrospinning. The scaffolds were successfully crosslinked by photo-polymerization using liquid or vapor form of PEG diacrylate for structure stability improvement. The characteristics in terms of morphology, fiber diameter, mechanical properties, porosity, permeability and swelling capability could be modulated by changing the composition of the scaffold. According to antimicrobial assay, silver-containing scaffolds could inhibit bacterial growth by 48 h. Silver could be released in a controlled manner over an extended period of time.

### Example 2

### Semi-Interpenetrating Fiber Scaffolds for Transbuccal Mucosa Drug Delivery

The oral buccal mucosa is a promising absorption site for drug administration because it is permeable, highly vascularized and allows ease of administration. However, there are barriers of macromolecule and polar compound transport between oral mucosal cells in the form of tight junctions. The tight junctions' structure and permeability are controlled by physicochemical factors such as the concentration of cyclic Adenosine Monophosphate (cAMP) and intracellular calcium. These epithelial barriers can be breached with the promotion of penetration enhancers which are classified into chemical and physical methodologies. Penetration enhancers are capable of decreasing the barrier properties of the mucosa by increasing cell membrane fluidity, extracting the structural intercellular and/or intracellular lipids, altering cellular proteins, or altering the mucus structure and rheology, in order to increase the permeation rate, without damage to, or irritation of the mucosa. Enhancer efficacy depends on the physicochemical properties of the drug, the administration site and the nature of the vehicle.

Penetration enhancers are thought to improve mucosal absorption by different mechanisms. For example, mucosal absorption is improved by reducing the viscosity and/or the viscosity of the mucus layer. Transiently altering the lipid bilayer membrane, overcoming the enzymatic barrier and increasing the thermodynamic activity of the permeant also improves mucosal absorption. Various chemicals have been explored as permeation enhancers across epithelial tissues. Among these chemicals are chelators, surfactants, bile salts, fatty acids and non-surfactants. Chitosan and its derivatives have also been extensively used to enhance permeation across either monostratified or pluristratified epithelia of small polar molecules and hydrophilic large molecules.

Through the mechanical penetration enhancers, drug absorption can also be enhanced mechanically, for example, by removing the outermost layers from epithelium to decrease the barrier thickness, or electrically, for example, by applying electrical fields or by sonophoresis. Such methods may be used in conjunction with the methods described herein. Applying electrical fields to the mucosal epithelium reduces the density of the lipids in the intercellular domain. As a result, intracellular pathways are opened, allowing substances to penetrate through the layer.

Outside of chemical enhancement, electrical mechanisms such as electrophoresis, electro-osmosis and electroporation are the most efficient permeation methods for solutes crossing the oral mucosal epithelium, and such methods may be used in conjunction with the methods described herein. Electrical enhancement for solute permeation is most effective for water soluble, ionized compounds. Electro-osmosis increases drug transport by using the inherent negative charge possessed in human tissue. These negative charges bind to available mobile, positive counter ions which form an electrically charged double layer in the tissue capillaries. When an electrical field is applied across the tissue, there is a net flow of water through the tissue in conjunction with the solvated counter ion. In electroporation, high potential (20-100 V) pulses are applied across the tissue. Due to the electrorestriction forces, cellular membranes are temporarily perforated or microchannels in the tissue are formed. These channels can serve as a drug transport route and are closed within a few minutes without any permanent damage to the tissue.

Drug delivery across the oral mucosa is designed to deliver the drug for either i) rapid drug release for immediate and quick action, ii) pulsatile release with rapid appearance of drug into systemic circulation and subsequent maintenance of drug concentration within therapeutic profile or iii) controlled release for an extended period of time. A proper balance must be struck, however, in the solubility and lipophilicity of the drugs. Other factors such as release kinetics can be controlled by the morphology and excipients of the polymer acting as the vehicle for drug delivery. The end products of drug delivery vehicles for the oral mucosa should be non-toxic, non-irritable, free from impurities and non-immunogenic. Lastly, to have effective drug delivery across the oral mucosa, drug delivery vehicles must have strong adhesive properties. The adhesion of the oral mucosa drug delivery systems must be able to rapidly attach to the mucosal surface and maintain a strong interaction to prevent displacement. Quick adhesion of the system at the target site can be achieved through bioadhesion promoters that use tethered polymers. The contact time is important because the longer that is, the more drugs are enabled for release at the target site. The bioadhesion of the drug delivery material should not be influenced by environmental factors such as pH of the oral mucosa. The drug vehicle must also possess the capability for high drug loading, complete drug release and convenient administration. Typically, drug release from a polymeric material and/or the electrospun scaffolds described herein takes place either through diffusion, polymer degradation or a combination of both. Polymer degradation can be done via hydrolysis, enzymes, bulk erosion or surface erosion.

There are many different formulations of drug delivery vehicles that have been developed as dosage forms. These include solutions, tablets (lyophilized and bioadhesive), chewing gum, solution sprays, laminated systems, patches, hydrogels, adhesive films, hollow fibers and microspheres. Advances in oral mucosal drug delivery have focused on the development of drug delivery systems that not only achieve the therapeutic aims of delivery but to overcome the unfavorable environmental conditions found in the oral cavity. Modern formulations have used approaches that incorporate a combination of these strategies in attempts to create a balance between patient convenience and clinical benefits.

Flexible adhesive patches were developed to overcome some of the drawbacks of the other dosage forms (e.g. tablets, chewing gum, etc.). Oral mucosa delivery patches have unique characteristics, including rapid onset of drug delivery, sustained drug release and rapid decline in the drug concentration once the patch is removed. Patches have the advantage of being more sustaining to deliver more drugs to the entire oral mucosa. Mucoadhesive patches are one type of patch which helps maintain an intimate and prolonged contact of the formulation with the oral mucosa allowing a longer duration for absorption. However, the disadvantage of patches is they only take up a small mucosal area and the backings have to be removed by the patient after being administered which reduces patient compliance.

In this example, we describe fabrication of a stable and biocompatible gelatin fiber scaffold for local drug delivery at the oral mucosa. To stabilize electrospun gelatin fibers and allow non-invasive incorporation of therapeutics into the scaffold, photo-reactive polyethylene-glycol (PEG)-diacrylate was employed to crosslink the scaffold to form semi-interpenetrating networks. The crosslinking parameters of the scaffold were systematically optimized. In particular, the concentration of PEG-diacrylate, amount of 2,2-dimethoxy-2-phenylaceto-phenone (DMPA) photoinitiator, and crosslinking incubation time parameters were adjusted. The resulting scaffolds were characterized by using a series of tests including scaffold morphology, tensile properties, porosity, swelling and in vitro degradation. The results confirmed that gelatin electrospun fiber scaffolds after being photo-crosslinked with PEG-diacrylate retain fiber morphology and show improved structural stability and mechanical properties commensurate with trans-mucosal delivery of active agents.

### Experimental Methodology

### Preparation of Electrospun Gelatin Fiber Scaffolds

The electrospun gelatin fiber scaffolds were prepared by first weighing out 1 gram of gelatin to be mixed with 10 mL of HFP. This mixture is contained in a reaction vessel and vortexed to help dissolve the gelatin into the HFP solvent. Lastly, the reaction vial is placed on a shaker plate and mixed thoroughly for 24 hours.

### Preparation of Gelatin-Drug Conjugates

### Electrospinning of Fibers

The electrospinning process has been well established fiber extrusion technique for textile and scaffold fabrication since the early 20^{th} century. To fabricate the fibers described in the present Example, a gelatin solution containing HFP was drawn up through a blunted needle of a 10 ml syringe. The syringe was loaded into a syringe pump which propelled the gelatin solution out of the needle 125 mm away from the collecting mandrel at a rate of 5 ml/hr. The needle was connected to a positive electrode of a high voltage power supply (Spellman CZE100R, Spellman High Voltage Electronics Corporation). The positive electrode contained a 25 kV voltage that was applied to the needle. This voltage helps the gelatin solution overcome the surface tension at the needle tip. These forces result in generating a Taylor cone which allows a steady stream of gelatin solution to flow from the needle to the grounded collecting plate in a jet-like fashion. As the gelatin solution is being streamed from the needle tip, the HFP solvent evaporates while in the air creating dry gelatin fibers. Randomly aligned nanofibers were collected on a flat, stainless steel mandrel 7.5 cm×2.5 cm×0.5 cm (L×W×T) rotating at ˜500 rpm within the translating electrospinning apparatus.

Once electrospinning was complete, the gelatin scaffolds were carefully removed from the steel mandrel using a razor blade, degassed under a film hood and stored in a dry environment overnight.

### Crosslinking Methodology of Scaffolds

Because gelatin is a very hydrophilic polymer vulnerable to dissolving in aqueous solutions, crosslinking is desirable to maintain mechanical stability. In this study, crosslinking solutions of PEG-diacrylate and DMPA photoinitiator in 2 ml ethanol solvent were prepared at different amounts. PEG-diacrylate was varied in 100 μl, 200 μl, 400 μl, and 800 μl amounts. DMPA photoinitiator was varied in 4 mg, 8 mg, 16 mg and 32 mg amounts. The crosslinking solution was poured onto the rectangular fiber scaffold and allowed to incubate for 30 min, 12 hours or 24 hours. The scaffold was then held under UV light for 2 minutes on each side.

### Characterization

### SEM Morphology and Fiber Diameter

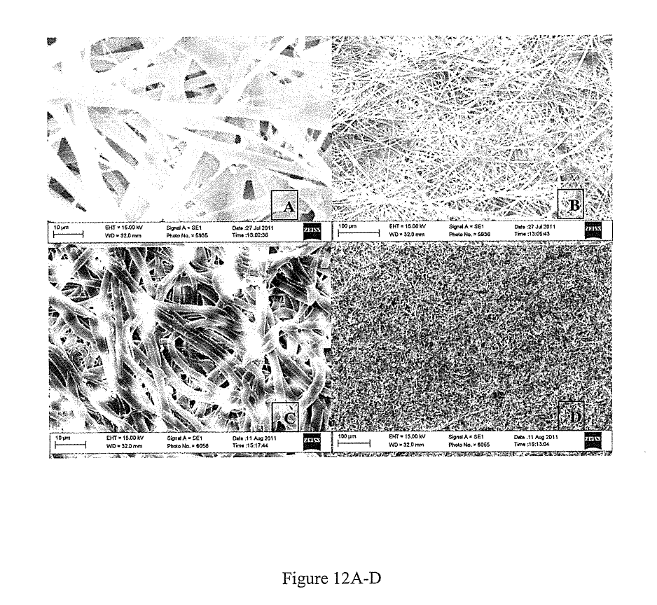

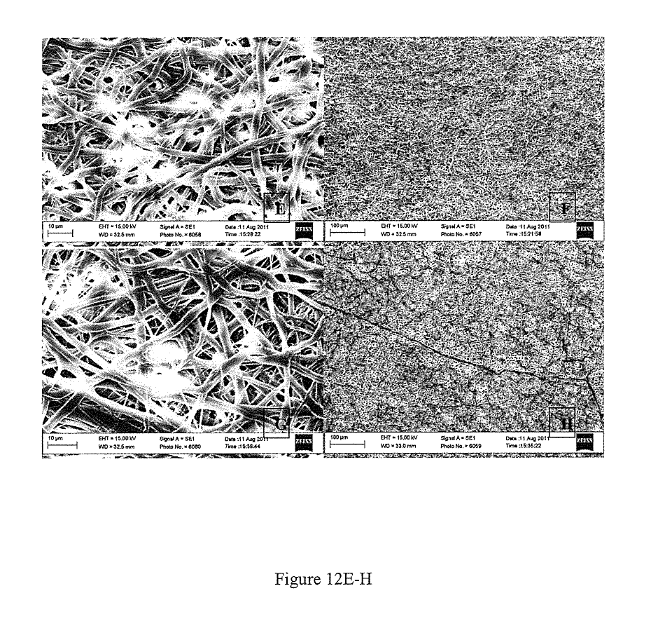

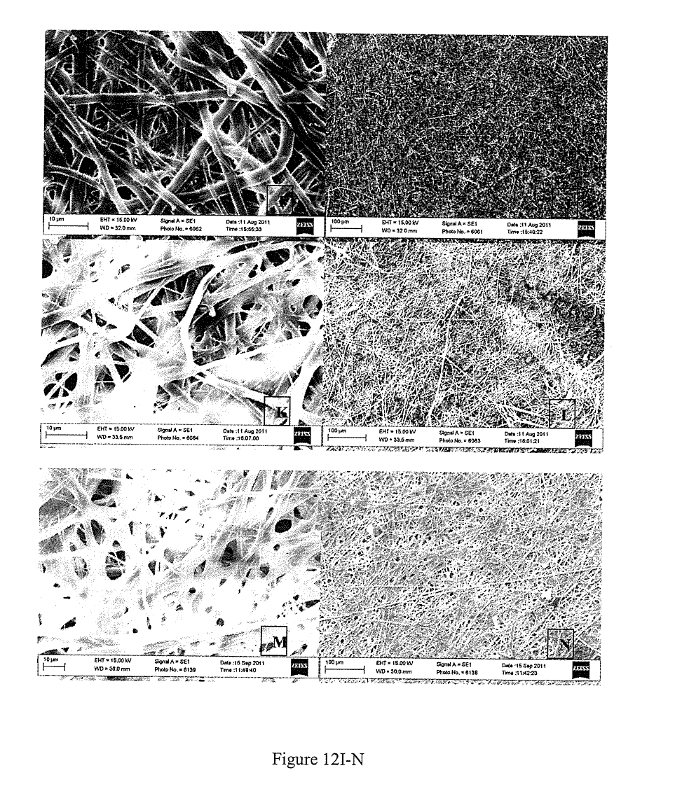

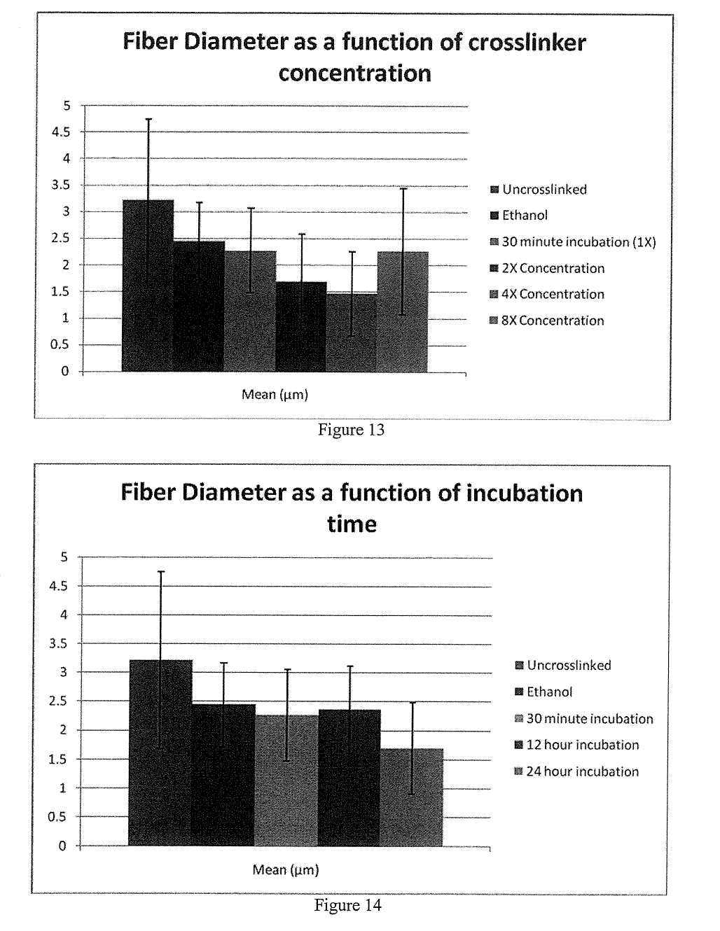

To analyze the SEM morphology and structure of the fiber scaffolds, they were cut into individually distinct shapes and placed on a 1 cm diameter stub. The stub was placed on a specimen holder and was gold sputter coated before being analyzed using a Zeiss EVO 50 XVP Scanning Electron Microscope at 10 μm and 100 μm bar scales. SEM images of uncrosslinked and crosslinked fibers are shown in FIG. 12A-M. Once the SEM images were compiled, UTHSCSA ImageTool™ software was used to measure 60 randomly chosen fiber diameters for each of the crosslinked scaffolds.

### Tensile Testing

The mechanical properties of the fiber scaffolds varied in crosslinking concentration and incubation time were tested using the MTS Bionix 200® Mechanical Testing System in conjunction with TestWorks 4.0 software. The fiber scaffolds to be examined were cut into 20 mm length dog-bone shapes whose narrowest point was 2.67 mm and gage length 7.5 mm. Ten dog-bone shaped scaffolds were tested for each crosslinking parameter to ensure proper statistical analysis. The scaffolds mechanical properties: thickness, peak load, peak stress, modulus, strain at break and energy to break were evaluated. The results fiber diameter is affected by crosslinker concentration (Table 6) and incubation time (Table 7).

TABLE 6
Fiber diameter as a function of crosslinker concentration

| | | | | | | 40% (v/v) PEG |
|---|---|---|---|---|---|---|
| | | | 5% (v/v) PEG | 10% (v/v) PEG | 20% (v/v) PEG | Diacrylate, |
| | | | Diacrylate, .2% | Diacrylate, .4% | Diacrylate, .8% | 1.6% (w/v) |
| Sample | Uncrosslinked | Ethanol | (w/v) DMPA | (w/v) DMPA | (w/v) DMPA | DMPA |
| Mean | 3.22 ± 1.53 | 2.45 ± 0.72 | 2.27 ± 0.79 | 1.69 ± 0.90 | 1.46 ± 0.79 | 2.26 ± 1.19 |
| (μm) | | | | | | |

TABLE 7
Fiber diameter as a function of incubation time.

| | | | 30 minute | 12 hour | 24 hour |
|---|---|---|---|---|---|
| Sample | Uncrosslinked | Ethanol | incubation | incubation | incubation |
| Mean (μm) | 3.22 ± 1.53 | 2.45 ± 0.72 | 2.27 ± 0.79 | 2.37 ± 0.75 | 1.7 ± 0.79 |

### Comparison of Tensile Testing

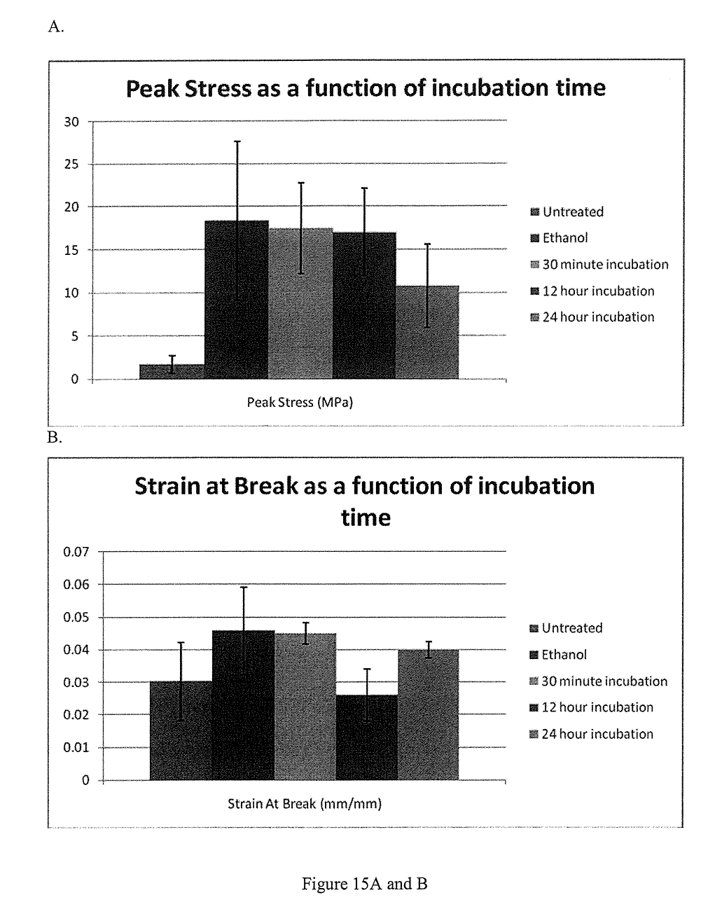

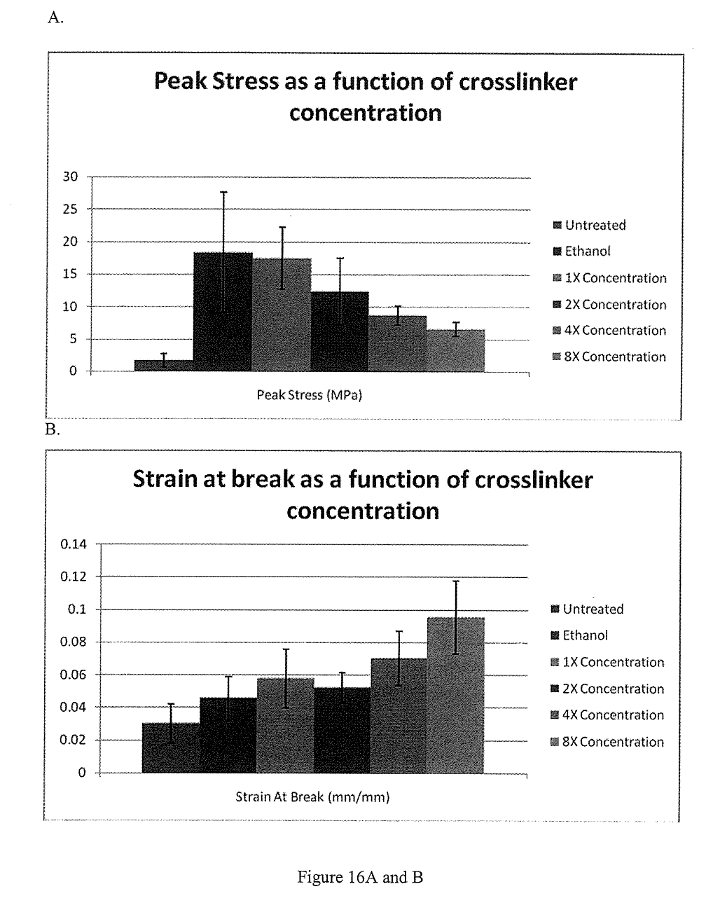

Tensile properties of the scaffolds were determined as a function of incubation time and concentration and the results are presented in Tables 8 and 9, respectively, and in FIGS. 15A and B and FIGS. 16A and B, respectively. The results showed that mechanical properties of the fabricated fiber scaffolds were also influenced by incubation time and crosslinker concentration.

TABLE 8
Tensile Properties as a Function of Incubation Time

| | | | | | Strain At | Energy to |
|---|---|---|---|---|---|---|
| | Thickness | Peak Load | Peak Stress | Modulus | Break | Break |
| Sample | (in) | (N) | (MPa) | (MPa) | (mm/mm) | (N * mm) |
| Untreated | 0.00915 ± .0012 | 1.0623 ± .5374 | 1.7589 ± 1.0036 | 94.2836 ± 41.4070 | 0.0286 ± .0120 | 0.1045 ± .0762 |
| Ethanol | 0.013 ± .0067 | 16.037 ± 6.7909 | 18.363 ± 9.2660 | 606.506 ± 201.9117 | 0.046 ± .0134 | 3.199 ± 1.9887 |
| 30 minute | 0.020 ± .0027 | 22.073 ± 5.4157 | 17.477 ± 4.7583 | 554.493 ± 153.8436 | 0.058 ± .0180 | 5.758 ± 1.8417 |
| incubation | | | | | | |
| 12 hour | 0.014 ± .0018 | 15.823 ± 3.9751 | 17.233 ± .8915 | 523.093 ± 153.5842 | 0.059 ± .0200 | 4.350 ± 2.1267 |
| incubation | | | | | | |
| 24 hour | 0.028 ± .0114 | 18.798 ± 6.0065 | 11.293 ± 4.8195 | 366.952 ± 203.7024 | 0.056 ± .0181 | 4.633 ± 2.2630 |
| incubation | | | | | | |

TABLE 9
Tensile Properties as a Function of crosslinker concentration

| | | | | | Strain At | Energy to |
|---|---|---|---|---|---|---|
| | Thickness | Peak Load | Peak Stress | | Break | Break |
| Sample | (in) | (N) | (MPa) | Modulus (MPa) | (mm/mm) | (N * mm) |
| Untreated | 0.00915 ± .0012 | 1.0623 ± .5374 | 1.7589 ± 1.0036 | 94.2836 ± 41.4070 | 0.0286 ± .0120 | 0.1045 ± .0762 |
| Ethanol | 0.013 ± .0067 | 16.037 ± 6.7909 | 18.363 ± 9.2660 | 606.506 ± 201.9117 | 0.046 ± .0134 | 3.199 ± 1.9887 |
| 5% (v/v) PEG | 0.020 ± .0067 | 22.073 ± 5.4157 | 17.477 ± 4.7583 | 554.493 ± 153.8436 | 0.058 ± .0180 | 5.758 ± 1.8417 |
| Diacrylate, .2% | | | | | | |
| (w/v) DMPA | | | | | | |
| 10% (v/v) PEG | 0.022 ± .0104 | 16.607 ± 5.2854 | 12.404 ± 5.1263 | 383.762 ± 196.7227 | 0.052 ± .0094 | 3.775 ± 2.0290 |
| Diacrylate, .4% | | | | | | |
| (w/v) DMPA | | | | | | |
| 20% (v/v) PEG | 0.022 ± .0043 | 12.438 ± 1.9219 | 8.715 ± 1.4404 | 251.021 ± 53.6256 | 0.070 ± .0166 | 4.242 ± 1.1114 |
| Diacrylate, .8% | | | | | | |
| (w/v) DMPA | | | | | | |
| 40% (v/v) PEG | 0.030 ± .0029 | 13.500 ± 1.8455 | 6.609 ± 1.0801 | 155.457 ± 30.3884 | 0.095 ± .0230 | 5.941 ± 1.9275 |
| Diacrylate, 1.6% | | | | | | |
| (w/v) DMPA | | | | | | |

### Comparison of In Vitro Degradation

The in vitro degradation of the different crosslinked scaffolds was evaluated using two different media in an incubator: i) incubation in Dulbecco's modified Eagle's medium (DMEM) at 37° C. ii) incubation in (DMEM) supplemented with 10% fetal bovine serum (FBS) at 37° C. and iii) incubation Simulated Saliva Fluid (SSF) at 37° C. Nine samples of 1 cm in diameter from each crosslinked fiber scaffold were weighed out and then individually immersed in a well of 48-well plate filled with 1.5 mL of either of the two aforementioned solutions for 24 hours. At 6 hr, 12 hr and 24 hr timepoints, three samples are taken out from the conditioned solutions and centrifuged for 20 minutes. After centrifugation, they are frozen, lypholized then weighed. The amount of weight loss due to degradation in each scaffold was calculated according to the following formula:

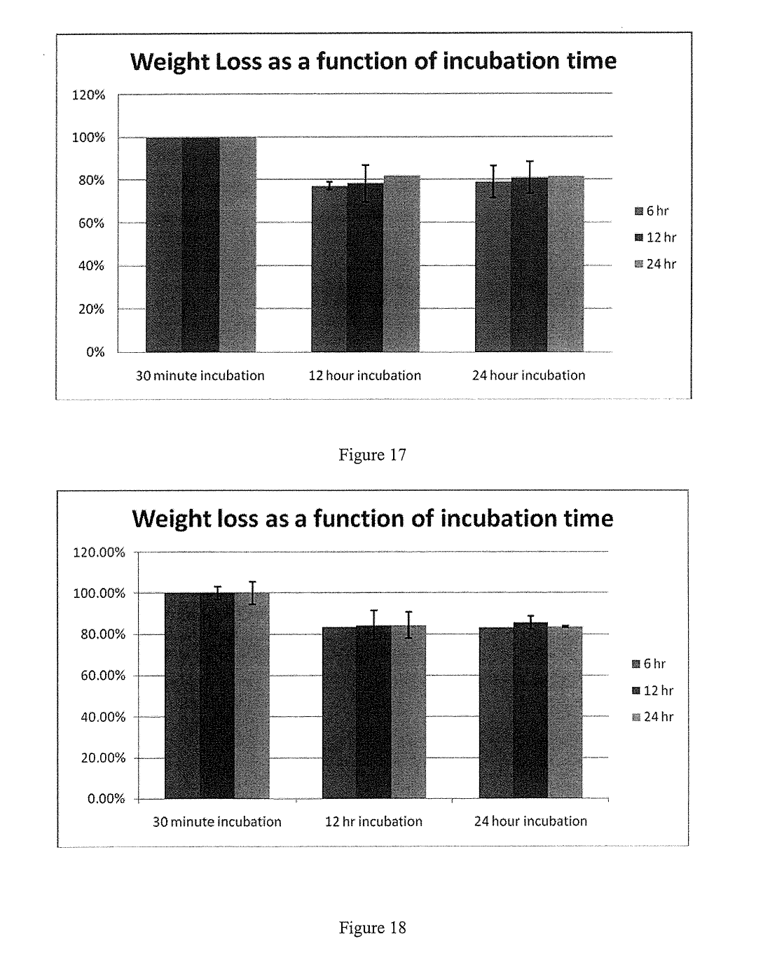

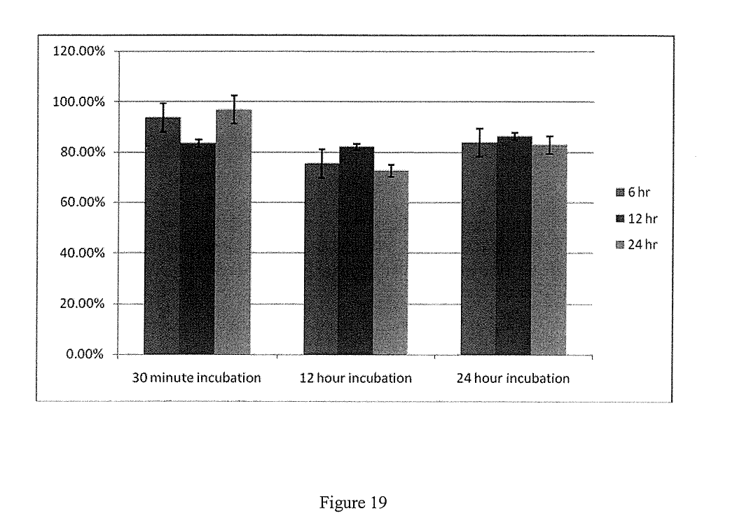

Weight loss due to degradation (%): [(Wₒ−W_{d})/Wₒ]×100
Wₒ=original weight of the sample
W_{d}=weight of the sample after degradation
   Degradation rates as a function of incubation time, as indicated by weight loss, were measured and the results are presented in Tables 10-12 and FIGS. 17-19. Degradation of the scaffolds were affected by medium as seen in Tables 10-12.

TABLE 10
In Vitro Degradation in DMEM +
10% FBS as a function of incubation time

| Mean Percentage | | | |
|---|---|---|---|
| Weight Loss | 30 minute | 12 hour | 24 hour |
| in Sample | incubation | incubation | incubation |
| 6 hours | 100% ± 0 | 77.10% ± 1.76% | 78.29% ± 7.44% |
| 12 hours | 100% ± 0 | 78.22% ± 8.51% | 81.05% ± 7.48% |
| 24 hours | 100% ± 0 | 81.94% ± 3.49% | 81.65% ± 5.88% |

TABLE 11
In Vitro Degradation in Simulated Salivary
Fluid as a function of incubation time

| Immersion time | 30 minute | 12 hour | 24 hour |
|---|---|---|---|
| in media | incubation | incubation | incubation |
| 6 hours | 100% ± 0 | 83.48% ± 3.03% | 83.25% ± 5.60% |
| 12 hours | 100% ± 0 | 84.32% ± 6.94% | 85.60% ± 6.38% |
| 24 hours | 100% ± 0 | 84.22% ± 3.13% | 83.53% ± 0.3% |

TABLE 12
In Vitro Degradation in DMEM control
as a function of incubation time

| Immersion time | 30 minute | 12 hour | 24 hour |
|---|---|---|---|
| in media | incubation | incubation | incubation |
| 6 hours | 93.69% ± 5.72% | 83.48% ± 5.72% | 83.25% ± 1.61% |
| 12 hours | 83.40% ± 1.45% | 84.32% ± 1.20% | 85.60% ± 2.43% |
| 24 hours | 96.80% ± 5.54% | 84.22% ± 1.38% | 83.53% ± 3.43% |

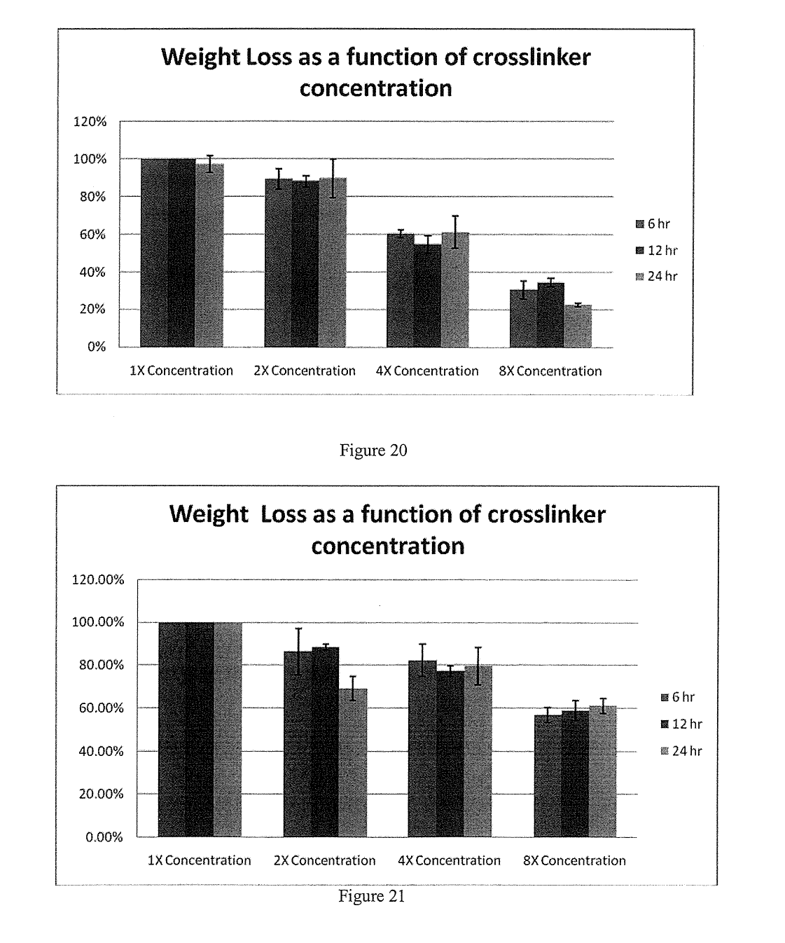

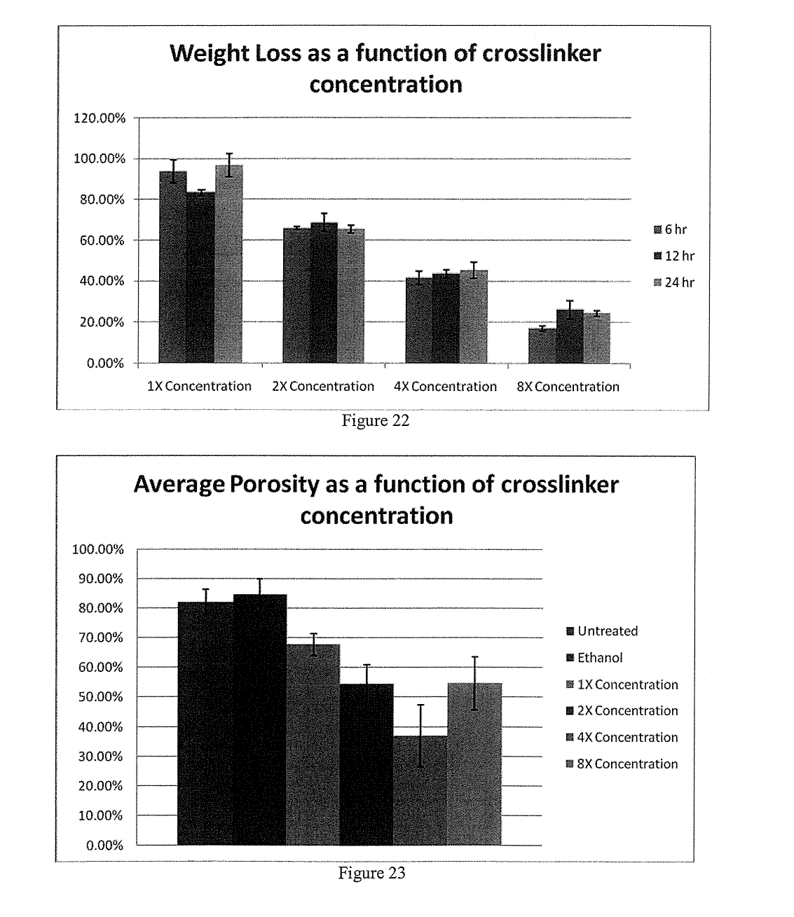

Degradation rates as a function of crosslinker concentration, as indicated by weight loss, were measured under a variety of conditions, and the results are presented in Tables 13-15 and in FIGS. 20-22.

TABLE 13
In Vitro Degradation in DMEM + 10% FBS as a function of crosslinker concentration

| | 5% (v/v) PEG | 10% (v/v) PEG | 20% (v/v) PEG | 40% (v/v) PEG |
|---|---|---|---|---|
| Mean Percentage | Diacrylate, .2% | Diacrylate, .4% | Diacrylate, .8% | Diacrylate, 1.6% |
| Weight Loss in Sample | (w/v) DMPA | (w/v) DMPA | (w/v) DMPA | (w/v) DMPA |
| 6 hours | 100% ± 0 | 89.25% ± 5.36% | 60.27% ± 2.01% | 30.63% ± 4.78% |
| 12 hours | 100% ± 0 | 87.98% ± 2.87% | 54.66% ± 4.63% | 34.59% ± 2.20% |
| 24 hours | 97.14% ± 4.50% | 89.74% ± 10.18% | 61.25% ± 8.54% | 33.41% ± 0.92% |

TABLE 14
In Vitro Degradation in Simulated Salivary Fluid as a function of crosslinker concentration

| | 5% (v/v) PEG | 10% (v/v) PEG | 20% (v/v) PEG | 40% (v/v) PEG |
|---|---|---|---|---|
| Mean Percentage | Diacrylate, .2% | Diacrylate, .4% | Diacrylate, .8% | Diacrylate, 1.6% |
| Weight Loss in Sample | (w/v) DMPA | (w/v) DMPA | (w/v) DMPA | (w/v) DMPA |
| 6 hours | 100% ± 0 | 86.31% ± 10.88% | 82.31% ± 7.70% | 56.75% ± 3.35% |
| 12 hours | 100% ± 0 | 88.55% ± 1.32% | 77.22% ± 2.34% | 58.82% ± 4.56% |
| 24 hours | 100% ± 0 | 69.10% ± 5.48% | 79.54% ± 8.76% | 61.12% ± 3.44% |

TABLE 15
In Vitro Degradation in DMEM control as a function of crosslinker concentration

| | 5% (v/v) PEG | 10% (v/v) PEG | 20% (v/v) PEG | 40% (v/v) PEG |
|---|---|---|---|---|
| Mean Percentage | Diacrylate, .2% | Diacrylate, .4% | Diacrylate, .8% | Diacrylate, 1.6% |
| Weight Loss in Sample | (w/v) DMPA | (w/v) DMPA | (w/v) DMPA | (w/v) DMPA |
| 6 hours | 93.69% ± 5.72% | 65.96% ± 0.75% | 41.55% ± 3.33% | 16.94% ± 1.19% |
| 12 hours | 83.40% ± 1.45% | 68.51% ± 4.43% | 43.47% ± 2.05% | 26.14% ± 4.39% |
| 24 hours | 96.80% ± 5.54% | 65.39% ± 1.89% | 45.36% ± 3.95% | 24.49% ± 1.39% |

### Porosity & Swelling

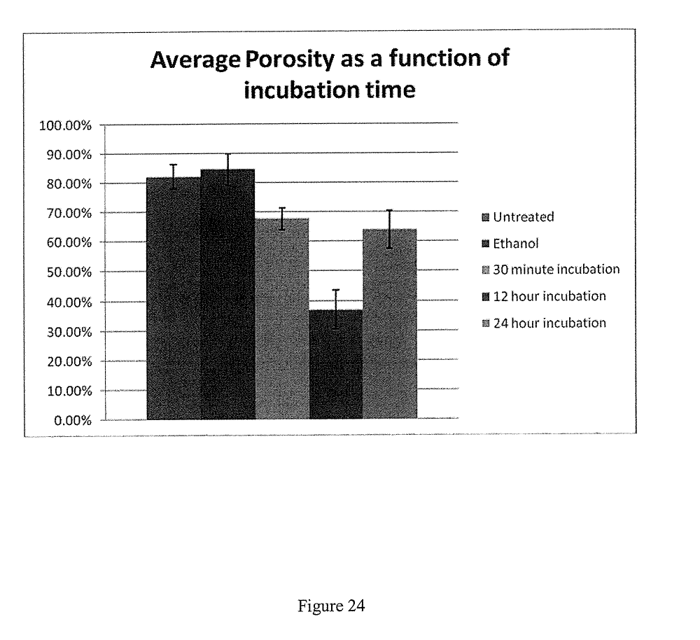

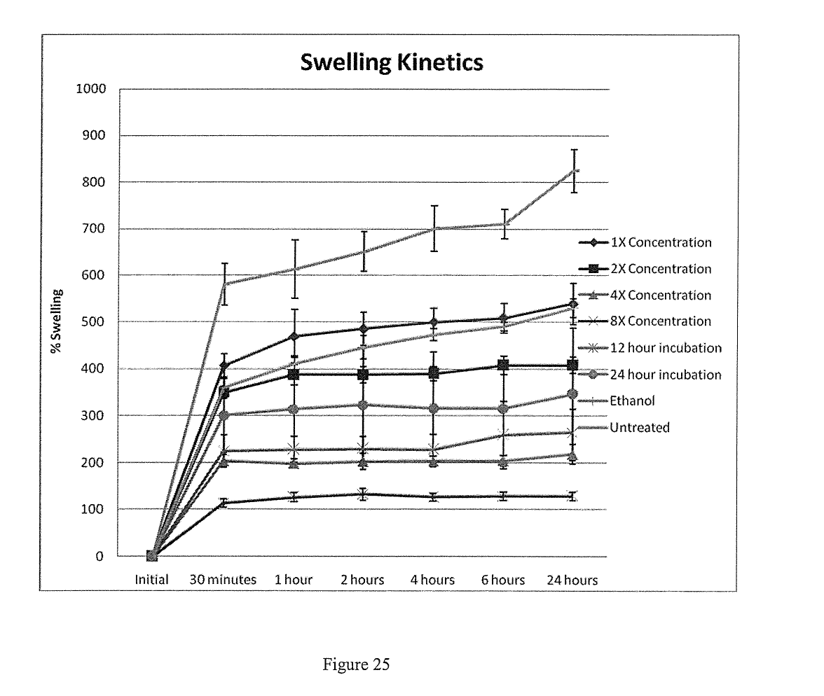

The results showed that porosity of the electrospun scaffold was affected by crosslinker concentration and incubation time. The results are presented in FIGS. 23 and 24, respectively. Similarly, the swelling kinetics of the scaffolds were affected by crosslinker concentration and incubation time (FIG. 25).

### Conclusions

The studies presented above show that it is possible to produce acrylate crosslinked electrospun gelatin scaffolds with properties that are commensurate with their use for the delivery of drug or therapeutic agents, particularly via the oral buccal route of administration.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

We claim:

1. An electrospun scaffold that is crosslinked with an acrylate.

2. The electrospun scaffold of claim 1, wherein said acrylate is polyethylene glycol (PEG) diacrylate.

3. The electrospun scaffold of claim 1, wherein said electrospun scaffold further comprises silver associated with said electrospun scaffold.

4. The electrospun scaffold of claim 1, wherein said electrospun scaffold comprises gelatin.

5. The electrospun scaffold of claim 1, wherein said electrospun scaffold comprises dendrimers.

6. The electrospun scaffold of claim 1, wherein said electrospun scaffold comprises at least one bioactive agent associated with said electrospun scaffold.

7. A material, comprising
electrospun fibers selected from a plurality of natural and synthetic fibers or blends, said plurality of fibers configured as a mat, wherein individual fibers within said plurality of electrospun fibers are crosslinked by an acrylate.

8. The material of claim 7, further comprising one or more dendrimers bonded to one or more fibers of said plurality of fibers.

9. The material of claim 7, further comprising at least one bioactive agent associated with said material.

10. A method of making a cross-linked fiber scaffold, comprising the steps of electrospinning a solution comprising at least one polymer to form a fiber scaffold;
associating a photoreactive acrylate with said fiber scaffold; and
activating said photoreactive acrylate by exposing said photoreactive acrylate to a source of radiation,
wherein said step of activating causes chemical crosslinking of fibers in said fiber scaffold via activated photoreactive acrylate.

11. The method of claim 10, further comprising the step of associating at least one biologically active agent with said fiber scaffold prior to said step of activating.

12. The method of claim 10, wherein said at least one polymer is selected from the group consisting of gelatin, at least one dendrimer, synthetic or natural polymers, and combinations thereof.

13. The method of claim 12, wherein said at least one polymer includes gelatin and at least one dendrimer.

14. The method of claim 12, wherein said at least one dendrimer is a polyamidoamine (PAMAM) dendrimer.

15. The method of claim 10, wherein said photoreactive acrylate is polyethylene glycol (PEG) diacrylate.

16. The method of claim 11, wherein said biologically active agent is selected from the group consisting of an antimicrobial agent, biologically active peptides and proteins, nucleic acids, drugs, cells, cytokines, lipids, antibodies, vectors, adhesives, permeation enhancers, metals, inorganic agents, imaging and contrast agents, or micro- or nano-particles.

17. The method of claim 16, wherein said nucleic acids include one or more of DNA, siRNA, and shRNA

18. The method of claim 16, wherein said micro- or nano-particles comprise an active agent.

19. A method of incorporating at least one biologically active agent into an electrospun scaffold, comprising the steps of
associating said at least one biologically active agent with said electrospun scaffold; and then
crosslinking said electrospun scaffold with acrylate.

20. A method of releasing at least one biologically active agent at a site in or on a subject in to need thereof, comprising the steps of
associating said at least one biologically active agent with an electrospun scaffold;
crosslinking said electrospun scaffold with acrylate; and
contacting said site with said crosslinked electrospun scaffold in a manner that permits release of said at least one biologically active agent at said site.
